SEQUENCE LISTING (9 sequences)

SEQ ID NO: 1 - DNA, 22105 bp, Homo sapiens
ggaaaaaaaaacagtccagcactcagaggaatggatgttaacagcctgagcagctcccaccccctcccacacaagaacagctccaaacatcattcctccttggactttcctcctccctgggcctacctcaaatgtacacatgtgaaacattcactaaacccgaagatgtctgcacacgacagctctattcatactcctcacccaacaaattccactctcctcaagaaagcctaggggtttgtgggaggtgggtctggcggcagaaggaaagcgaggtcacggagaccacatccctgaatgatgcctggctcgactcaaacgcaacacgcgtgaatgctagggcaggctgctcctgcgcggccctcctccacggagtgccagggagggagcgcaaagccaccagcgcggcggagctggtgcgcctccggcccgcctctccggtaacgggtcgcgccgcactgagtccttgcagcaaacctgccgaatcccgcccgtggacctctaaacaccgggcggggccgggtgtggcggcgcgctcggacgttcgtcattcctccgcgccctccgcctgccagctgattggcccctgggccaggggcggggcactcgcggcggaggcaagcggcggcgcgcggacggttggtccagttctccggcctggcggcaggcaagtctagctcggcgctgtcggatacttggggtgagcggaaagcatggcggggacctccgcgccaggcagcaagaggcggagcgagcccccggcgcctcgccccggcccgccgccgggcaccgggcaccccccgagcaagcgggcccggggcttctccgcagccgctgccccggaccctgacgacccgttcggcgcgcatggggacttcactgccgacgacctggaggagcttgacaccctcgcgtcacaggccctgagccaatgtccggccgcggctcgggacgtgtccagtgagtgctcctcgcggccttttgctcggagggagttgtcaaccgcgccagatccccttgatggctgtggcttcggaacctcgcggccagcactgccttttcgcctttttaaaatatgggaacaccctgatttaagcagcggttgtcttccagaaggtcctttgattttagggggaaatgcattagccaggtcaaacagccgatttgaaacacagaaggctaagttgacattttacgttattttctaaagtttaggccacttggttcttggttctaagcagagatccttggaacacacctaccaccaccatcagaaccatcactacccatgcatggggacccattcttgtgacaagtttgagcggctttagatcttatgaagcccactgtctcccttgggcagtgtgagcatgggaggagccagacacaggcaagagatgaaataaatttattcttttatacaaccagtaaactgtgctaggtgcccaaacgtgaggaactgcagctgaccttacaatgaacaaaatacaaaaactgtccttcatggagcttacattctagttgaggagtgagtgaagtcttgaggactcccagaaagagaaatgttttgagtgcaccttgcatctgtacttgggtgtctcaaggcaccttaaactcaacacgatcatttcctggaaacccagtcctcctccagcacttctagcatggtgccggcaaagcagacattcagagaatgatgaccagatgttccacaaggaagtaactagcacgactctccatccagtggcacaaagcgactcttcccctttccttgactctacatctaatttatttggaaagcatgtcaagcttaccttcttaatccttctctggagtctatcctattcacattgcttctaccagctgcctgatctgtgttaccatgagtcctctcttgaacaatggtctgctaaattggtttcccatttccagtcttgactccctacaagtgaggcttcacatcacattctgagtgatcttttcaaaactcaaatctggctgtgtcttacacagatatacgtgaacaccctttttttttttttttttttttgacggagtctcactctgtcacccaggctgaagtgcagtgatgccatcttggctcaccacagcctctacctcccaggttcaagagattctcttgcctcaacctcccgagtagcctcagcctcccaagtagctgggattacttgtgcccgccaccatgcctggctaatttttgtatttttagtagagacggggtttcgccatgttggccaggctggtctcaaactgctgacctcaggtgatccacccgccttggtctcccaaagtgctgagattacaggtatgagccatccgcacccagccatgaacacccctaatgaaagccctttaataatttcttatttcttctagtgtgaagaccagaatccttaacatggcctgaaaggcccagcctggtttggccctttctcactctctgggtcctggctgcactaacattctttgaattctctctgcttctttacaatacaggaactttatacattgttctttctttgtacattgagtatacttttcattcattcatttccactcatctggttaattcctacttgtccttcagctctcagctccttcaagtcttccctagggaaatgttccaatcatcccagttaagtcagattgctgtcatattataggctttcctatatttgttctcttctgcagttcttaatcacagttgtaattttacatttatctgtgtgattatttgttgaatgtcttttaagctctgtgagtgtaaggaagtatcaattttgctcactattgtacctgtagagcctaacatgcctggcatattgcatgtggtcagtaactattactgaataataagtagatcattgagttctggccaatgagatctcctcaggctttagatgagactcttggaattggatgaaggatagggaaagaataataggatctgttgttttagaaggatcatttatgagtttcagccgggtgtggtggctcatgcctgtaatcccagcactttgggaggccaaggtgggctgatcacttgaggccaggagtttgagaccaggctggccaatatggtgaaaccccgtctctactaaaaatataaaaattagccgggcgtggtagcacacgcctgtaattccagctacctaggagggtgaggcacaagaatcgcttgaacctgggaggtggaggttgcagtgagccgagatcacgccactgctctccagcctgggcaacagagcgagactctgtctcaaaaaaaaaaaaaaaaaaaaaaaaaggatcatttatgagtttgaaccatgttcttaggttgaaaaacatctatattgcttactagatctttccctgctttgctctcaagagtaattcttcccagcactttgggaggcaaaggcaggaggattgcttaagcccaggagttcaagaccaacctgggcaacataaggagacccaatctctacaaagaattttaaaattagccaggcatccaggcacagtggctgacgcctgtaatcccaacactttgggaggctgaggcgggtggatcgcgaggacaggaaatcaagaccagcctggctaacatggtgaaacgccatctctactaaaatccaaaaaaaaaaaaaaaaaaaaagccaggcatggtggcacacacctgtagtcccagctacacaggaggctaaggcaggaggatcacttgaacccaggaggtcaaggtggcagtaagcagtgatcacaccactgcactccaacctgggtaacagagcaagaccctatctcaaaaaagaaaaaaaaaaaaaaagtgggtattttcagcattttctggcaaaagtgggaaaatattgggtcctgaaatgtatatggaactattttttacaggtgatcataaggtccacagattattagatggcatgtcaaaaaatccttcagggaaaaacagagaaactgttccaattaaagataatttcgaattagaggtacttcaggcacaatacaaagaacttaaagaaaaggtaagtgacttaacttgtggtttttgtagctaattcattcacttacgtgtttaaaagcattgtacagtaaaatatacaaaagttgtaaaacaagtgtctagattcatccctatgacatgggaaaagactagggaaagaaaaccagaaagtttaggctgaaagaaactaaattgtgagattcctaatgccaaaggcaagaagaaaaacactgtataccagatccaaaaagagatacaaactgttagggtactaagatgaatttatagcatgtatcttcatgtgaataatacaacttcataatggttgctattttcattagcagctttgcaagaaagcagacacttcatatcaccatctgtaggtccctaagaaaaccaggaatatgtggaattttagttgtgcaaggtatgtgacccagattttttttttttttttttttggagatggagtctgtcatccaggctggagtacagtgacacaatcttggctcacttcaacctctgcctcctggttttaagcgattcacatgccttagcttccagagtagctgggactacaggtgtgcaccacgacgcccagctaattcttgtatttttagtagagacggagtttcaccatgttggccaggctggtctcaaactcctggcctcagcctcccaaagtgttgggattacaggcatgagctaccacacccagcgacccagatcgttaatgcctcttagagcctaaagaatccggccaggcgcggtggctcacacctgtaatcccagcactttggtaggccgaggccagtggatcacctgaggtcaggagttcaagaccagcctggccaacatggtgaaaacccgtctctactaaaaatacaaaaattagctaggcatggtggcgggcacctgtaatctcagctactcgggaggctgaggcaggagaattgctggaacctgggaggtgaaggttgcagtgaaccgagatcgtgccattgcactccagcctaggcgacaatagcaagactccgtctcaaaaaaaaaaaggaatctggtcaggcaaggtggctcatgcctgtaatcccagcactttgggaggccaaggtgagagaatcgcttgaacccaagagttcaagaccagcctgggcaatacaatgagaccctgtctctacagataaaagtgaaaattggccaggctagtggcacatgctttgtagtcctagctactcaagaggttgaggcgagaggatcacttgagcccaggagttcaaggttacattgagctatgattgtgccacagcattccagcctaagcaaccccatccccctccctggcaaaaagaatctgcatgcatgagtggcaagtgtatgcttaagctatcttgatgaatatcagctgtcccatgaacaggcctagcagaccatggtgacatctggttgctgtggatatattttgagtactcatttgtctgtttttcctgtgctttctccatctccccaactacatgttaaaacaaagtacctaagtagttttctcttacaaagttttcacgagattaatttgggatttacagatgaaagtaatggaagaagaagttctcattaagaatggagaaattaaaattttgcgagactcactacatcagacggaatccgttctagaggaacagagaagatcacattttcttcttgagcaagagaaaacccaagcactcagtgacaaggaaaaggaattctccaaaaaggtgacccagagcatttgttttgcaccagctttgcctgcccactgagttcctttgaccagggttgcctgtaaatcttccagggagatttcaacacttgtttgtcttaaatactttctgctatcatctcattgccatccactcttcttccagggtctggatatattttggaaagggatttagatgaaactctattttgctgtggtagttggtcttggcaggagtaaataactagcttagcatttctagaaacaggtttacattaaggctcttttgctagatcagaaactcaactgacctggcttaggctaaaaggaaacgtattggcttccatatccaagtctgggaagaaccggggtatgctgggaccttaaatgacttaaatcttgccaggacatttttattctctatgctataaacaggctctatacttatggtagagggtgtgttctccagaagctcctgggctcatattttataaatacagagcagaattaagacttagggccacatgcagtggctcacacctataatccagcactttgggaggctgaggtgagaggattacttgagccaggagttcgagaccagcctgggcaactagtgagacctcatctccacaaaaaagaaaaagaaaaaatgaacaaaattatccaggcatggtggtgtgcacctgtagtcccagctactcaggaggctgaggtgggagaattgcttgagcccaggaggttgaggctgcagtaagccatgatcatgccattgtactccagcctgggcaatagagcgagaccctgtctcaaaaaaagacttaggctccctcatctcaagttttaaaattctgagaaaggctctgattggtttaacttgagtcagttgtctgtccctggccaattgctataaccaaaagcaaccaaaagcaaaaaaatgacagctcccattctaataaaatagtcagaggtagggaaattattgaatttttttttttttttttttgagacagggtccagggtttcactctcttgcccaggctggagtgcagtggtgcaatcacagctcactgcagccttgacctcccaggctcaagcaatcctcccatctcagcctcccaggtagctggaactataggcacacaccaccaggcaaagctaatttttaaattttggtacagacagggttttgctgtattctccaagccgttctcaagctcctgggctcatgcagtcctcccaccttggccttccaaagtgctgagattacaggcgtgagccacaatgcccagcctgtttctcagggaaaggaaaatgctattctggaaggaaaaacagtaggtatccagtacctactacaactttccctcagcacctcctcatgcttcaatctcaaggacatgggatcttattctcattgtttcattcagcaaacttagcaaactttaagcacttacctgtgcccatcattaaccgcaatagttaactttctgtataagtgatgaagagcaaggaatttgcggtcagacaaacctgaatttgaatccaggcttcatcacacataagcagtgacctcaaggtaataacttaagctccctggctcatttcccttatctgttccaagacataaccagaaaggttgttgggagactcacacattcttgcacttactgtgtctggtgctgggcctggcgcagagtgtgcttggggaatgatggcagttactgttggctgtagcaactgcaggccagagcagcaggtctctctatactgcacagtgtcttggtaaactattccaactactctgcccaaactttgaggaagacctggaattgtgcaccagtgcgttcgttaataaaggtcaatatgttactgctgacatagggcaccttaccaagctctatgctgggtattttacactcatcattctacttaatcctcccaactatcatgaagacaggtgttttatgttacaaataagttaggattcaaagacattaaataaatggtgggctggtatttaattcttttttttattttttaaattttttgtagaggcaagatcttgctattttgcccaggctggtctcgaactcctgggctcaaatgatcctcccgcctcaacctcccaaagtgcttaggattacaggcctgaaccaccacacccggccagggctggtatttaaaccctcacaaactacccttacaatagagtccagtatctttttagagtaccatattgtctttcagaaagaaagctaatgtttaatattttaggacatttatgtcacagcagactttcatatgtatttttcattgtttgtcttttactggcttgtgattttgaagcccagagatatttttgtgtatatgatgggaccactacaagcatgtttcttttgccttccagctccaatcattgcagtctgaactccagtttaaagatgcagagatgaatgaattaaggacaaagctccagaccagtgaacgagcaaataaactggctgctccctctgtttcccatgtcaggtaatgatggtgctggagggatagttcagttttgcattatttagtaaaaaatctgcataacagtgtcaggctggtcctaaatgaaggccatttagacccctccacctgggcagagatcctcttttcctgaaggattcagaggtggtttagatgaagcttgcatggtttgagacatcacagcataaaggcctatactctagaaatgctgcctctgtatggcactttataaacataattcttcaggactagaaagacctggaaaaatgatctccaaattttgggggtgggctagtctctacttttatgtaatttgtttaactgattttgttgcaagcatgggtgtattttagttccagctacttggaatatttttttctcatttgctttctggggacccttgtccaacagccccatcctttgtggtcagatgtgctcaacttgttttttttttcttttttttttgagacagagtctcgctctgtcacccaggctggagtacagtggtacaatctcggctcactgcaacctccgcctcctgggttcaagcaattctcctccttcagcctcctgagtagctgggactacaggcacacaccaccacgcccagctaatttttgtatttttagtagagacggagtttcaccatgttggccaggctcgtctcaaactcctggactcaaatgatttgccagcctgggcctcccaaactactgggattacaagcatgagccactgtgcctggcgcagcctggaaatttgaacagtgtcagtcagggcatcttctggttttgaactttctaccgcctttgttcataatgggcagtgtgtgaaactgaactctccatgccttgcattgtccacataagacaagagaactgaggggatcctcttcacacatcagtaacagaaagacctcagcattatatgatcagatcagagggttaatactgcttctctaccataaagacattggcactaagttgctcagttttcaacatacctagtaggtttatcaaaattatgatatttattttgatagccctttttctatttaaagtctttgcattctattggaagtcttttttttttgagacagagtctcactctgttgtccaggctggagtgtagtcacatgatctcagctcactgcaacctccacctcccgggttcaagtgattctcctgtctcagcctcccaagtagctgggattacaggcacataccaccacaccaggctagtttttttgtattttttttttaagtagagatgggatttcaccatgttggccaagctgctctcgaactcctgacttcaggtgatccacccgcctcagcctcccaaattgttgggattacaagggtgagccactgtgcccagcctggaagtcttgtgactattaaaaaatcgtttgttccacattggctgggtgcagtggctcacacctgtaatcccaacactttgggaggatgaggcgggcagatcacttgaggtcagtagttcaagaccaacctggccaacatgatgaaaccctgtctctactgaaaatataaaaattagctgggcatggtggcatgcgcctgtaatcccagctatttgggaggctgaggcaggagaatcactcaaacctggaggcagaggttgcagtgagccaggatcacaccactgcactccagcctgggcaacagaacaagactccattccagaaaaaaaaatagtttgttccacattgaaaagatgttttgcaccaggcatggtagctacagaggcaatcccagctacacaggaggctaaggcaggaagattgcttaaggctagcagttcgagaccagcctgggcaacatagcaaaaccccattgctacttaaaaaaaaaataaaattactggccccagtggctcacgcctgtaatcccagcattttgggaagctgaggtgggcagatcatttgagcccacgagttcaggactagcctaggcaacatggtgaaactctatctctagaaataactctatctctagaaagaataaaaagttagccagatgtggtggttgcacatctgtagtccctgtgctactcagaaggatgaggtgggaagatcactaagcccaagaggtggaggctgcagtgagccgtaattgtgccactgggctccagcatgggcaacagagtgagaccctgtctcaccaaaaaaaaaaaaaaaaaattagctggccatgttgatgtgtgcctgtaatcccagctactcaggaggctgggattacagatgggtggattgcttgagcccaggaggtcaaggctgcagtgagctatgatcatgctactgtactccagcctgggcaatggaacaagactctgtctcaaaaaaaaatgttttggtggaattgtaaatagtgataataaagaatagtaaaggaattatggctaatattatatttttggatgattagaaaaatataaaaatgagtaaaagaattttcaacaatttggatcaaaatccctggaattagccagtaacagtggatcatgcctgtaatcccagcattttgggaggccagagtgggtggatcacttgagctcagcagttccagaccagcctgggtaacttagtgaatccccatctctatagagaaattaaaatttaaaaatatttttaaaaagaaaaacaaatgcctggaattttgcactgctcctctgacaaagaatggtagacatataatagataaaacctttggttaaaacacctagacatgaaaagtttaaagtttgttagaaattttgtaaccagatattttgaatggttttagtagaatcattactttgctttcatagttaactttttccagtcctaggaaaaacccttctgtggttataaagccagaagcatgttctccacaatttggaaaaacatcttttcctacaaaggagtcttttagtgctaacatgtcccttccccacccctgccagacggagtcaggatacaagcctctggtgggcagagagggtaagtccattagtcatctattgatgtataacaagctacccaaatatgtggcttaaaacaattattatttattttctcccaatttctgtgatcagcaattctgacaaggcacagaggggacaatctctgcagggactggaatcactgaaagcttgtcttaagctggaggatctatttcaaggtggctcactcacatggctggcagtgtaatttcttgccacatgggcctctgcacatggatatggggtatcctcttaacctggtgggtggcttcctctaaagctagcaaatccagagagagcgagccaggcagaagctatccatttatggcctagcctcagaagtcacacagcatcccctctgccacattgtatttgttagaagcaaatcactgagtctgggcgtcattcaaggggagaaaccaagcttataccttttttttaatttttattttttatttttattttattattatactttaaattttagggtacatgtgcacaacgtgcaggtgtgttacgtatgtatacatgtgccatgttggtgtgttgcacccattaactcgtcatttagcattaggtatatctcctaatgctatccctcccccctcctcccaagcttataccttttgaaggggagttgtgtcaaagcatttgtgggcctttttttttttttttttgaggcggagtctcaccctgtcacccaggctggagtgcaatggcacgatctcagctcactgcaacttccgcctcccgggttcaagtgattctcctgcctcagactcccgaatagctgggattacaggcacctgccatcatgcttgtgggcatatttttaaaccatcacagcatcaattgaccatgccaaaaacctagctagtatgcaggaatctgtttttcttttcttgtttttttttttttctttgaggcggagtctcactctgtcaccaggctggagtgcagtggcgtgatctcggctcactgtaacctctgcctcccatgttcaagcgattctcctgcctcagcctcccgagtagctgggactacaggcatgcgccaccacacccagctaatttttgtatttttagtagagacaggtttcaccatgttggcctggatggtctcaatctcatgacctcgtgatccacccacttcagcctcccaaaatgctgggattacaggcatgagccaccgtgcccagcctctgttttccttttgtagtccagcttttgagtttttataactcagtccagattgaggggatctgctgttttttagagagagacaatctaacatcagtaggttttcagactgctatgtcagtgtatcttataaatcggatacattgaccctcttcttaccatcaacctttcaaatccttggccatcataacacttctgagaaactgtagcccacttacatttttggcatctagcagctaaaggggcagtgtacagtagtgttacaagcgggcagtagagcccagtagcctggatgtgagtcccagtaatgctgttaactggcttatatgatttgggcaagtcctttagtctctgtgcctcagtttccctatatgtaaaacagacctaatgataaatccttgtagggtcactgaggaacaaatgggctgatgcgtatagagctcttaaaacactgtctagcacgtaacaagcaccatgtcaatgttagtggtcactgttttatctaattgcctcattttgagtgaggcaaacagcttgattttcttttcctttccttgtatgagttcctgttttacataaggaaggtaaaagatgcagggctcttgagccgctttggctcttgtttgttccaagttcgaagaagtagaacagcgtgtgttttaaactcattgcatgtaaaagtttctaatatgcccatgcttctgggctcaagttttgatttacattttatcacatttcagatagtaagccccacagtctgagaggtgactccataaaacaagaagaggcccagaaaagctttgttgacagctggagacagagatcaaacactcaaggtaccagaatccctgcttctcctgcaggggcctgcatggctctgagtaatatgcagaagaattgcccaggcctctgagaaccggtgccccgggcagtccttcagagagtccagggaagtcaaagtatgtggcttaagcagaagactttataaagtggaaagggccaagggcaggtatgtcccagagccttccgcacccacaggcatcttcctggggcttagtggcagcctagaggacagttggcatccaaagaatccttactgtttccttctgaaagccaccgaaaagatctcccatgtcagaaccttctagagtcatcttgtcttctgcaggttccattttgataaacctgctcctgaagcagcctttgatcccagggtcatccctaagcctttgccacctcctgagtagtagttctgagtctcctgctggcacccccctgcagccaccagggtttggcaggtaagcataagactcctggaaagcttgtttgggaaggagcttcctggaagcaaattcctttctggccctggccagcctgagaagtctgtgggaagggcaggcagcctggagaatttggcaggcaggctgtttggggctcttgaccttatctccatcccacacttaatctatttttctttgtgtttgttgccagtaccttggctggaatgtcaggcctcaggaccacaggttcttatgatgggtcattttccctctcagccctgagagaagcacagaacctggcattcactggactgaatctggttgcccggaatgagtgctcacgtgatggagacccagcagagggaggcagaagggccttcccactctgccagcttcctggagccgtgcatttcctcccccttgtacagttcttcatcggcttacactgccaggccctgcaggacttggcagctgctaagagaagcggagcacctggggactcaccgacacattcctcctgcgtgagctctggggtagagaccaaccctgaggactcagtgtgcatcctggaaggcttctctgtgactgcacttagcattcttcagcacctggtgtgccacagcggagcagtcgtctccctattactgtcaggagtgggggcagattctgctgctggggaaggaaacaggagcctggttcacaggcttagtgatggagatatgacctcagccctaaggggggttgctgatgaccaaggacagcacccactgttgaagatgcttcttcacctgttggctttctcttctgcagcaacaggtcaccttcaagccagtgtcctgacccagtgccttaaggttttggtgaaattagccgaaaacacttcctgtgatttcttgcccaggtattaagctgcataggagtcatgattctttgtgggtctcacctgacctcttaaggtctaacccatggcacttgtactttgctcacatcttgacttatctacactagttagttctaaggcttggttcagggagttctgaagtgatgggaagtggggccaaatgggagaaccaaggaagtggtttatttgatttaattctgcctctgaacaacaggtaagcaaaaggcacacatgattaaaggaggctgctaacctctgtggaggagggccctgccccaggacaagggttaagacaaggggcctctgaacctgagatgtgggtaaggtgaagaccacacatcacaagcacgttgaagtttagtggtttccctaaggaagcagaggatgtggagagggtctgcagcccacgggctgaccttgctgggagtctctgcctgtcattgtgcccatctttagcatgcatctggggtggcctgttcttcttaaggtttgtgggtagtatattatagtgaacctagcgaatgaacactgtgatcataaattcctcccttgggattccagagccgcaagattggggagaactgtgaacacaaccagcccaccccctcatccagtaatccagtctccctcagcactctaccaaagggtcagtttctatgttggaatgattccactggctgaatatatcttaaatcatctcatctctgagctcagcactgctgtttcctagatctgtttccatctgaatatttttggttggtctggcccatgctaaaggtggtactcagctggacacagcactgcagtgtagtgtgggcagtgggatcaggcagactcaggactcaagtgcccagcaggggcccagctcatagtggctgtttttttttgttgttgtttatttgtttgtttttgtttttgttttgagacgaagttttgctcttgttgtccaagctggagtgcagtggtgcgatctcggctcactgcaacctccacttcccaggttcaagcagttctcctgcctcagcctcctgagacgctgagattacaggcacctgccaccacacctggctaattttttatatttttagtagagacagggtttcaccatgttggccaggcttgtctcaaatgccggacctcaggtgatccacctgccttggcctgccaaagtgcaaggattacaggcgtgagccaccgggcccagccttggttatttgtttaataattgttaaatgaatgaatcccaggctgggcacagtggctcctgcctgtaatctcagcactttgggaggccaaggtgggaggatcacttgagcccaggagtttgagaccagcgtgggcaacaaagcaagactcccagctctacaatttttttttttttaattagccaggcatggtggcagtgagctatgaccatgccactgcactccagcctaggtgacagagtgagaccctatctcaaagaatcccagaaaccatgtttttactggtggtagacacaccattattccactcctgttgagcttgtccacagggacattcaaaacacagccacagctgggcatttatctatttcatgtactggggctcttcataaaatttcatttaaagaggttcttaggggctgggcatggtggctcacgcctgtaatcccagcactttaggaagccgaggtgggtggatcacgaggtcaggagatcgagaccatcctggctaacatggtgaaaccccgtctctactaaaaatacaaaaaagtagctgggcgtggtggcaggcacctgtagtcccagctactcgggaggctgaggcaggagaatcgcttgaaccctggaggcggaggttgcagtgagctgagtttgcgccactgcactccagcctgggtgacagagcgagactcagtttcaaaaaaaaaaagttcttaacgctatcaaaaaataacttggaaaccactgtggtgtacataaaagtggattttccttgcagtaccttctgcaagtggcagatattggacatcttacagcccaataatgaaaccaggtaataaccatgttaaaattccagcttcccacaaagggctgcacaccctggcaactaaaaagcattgaactttttgtctgctgtgcaggatttgccctaaagtgagttcaccccacatagtgagaccttgcttcctaaggacacactgagggatatcttgaccttctgtgcttagaaactgcagatgtctgaaggggtacacacctggccagaccccatagagtgtttcagaattgccctcctccccacccaccaggccagtccatcgtgctcccccaggctatgtgggttgagcaaacagcagcagttgccttggggcttggtttattggggtaatagctttccttttgcccctgagacctggggcagtatgcccagaacccctccctcctgcacaccccacagtaggcacattctcccactggcagcagatgtgttttctggcaagtagagaaaatggctttccttgccctgtgagaggtctcataagatccagccatagccaaggccatgtttgtcacctctggaggcctggtcagtccttctgtgggcacagaacatttctctcccacctaagttaggacctgcatccctgtttgggagcatgtggaagccaggcatcccagatccaggaggtcatcccacttcctgggcgatgctacaccagataagggcctgtcctctcagctgcagcagtcttgcagtggaagtgcccacttgttcacatgaaacaagtttcatagcaagtaagtaggggtcaggccagcactgaaacccaagtctggctgaaccaactcacctgttcatactgacttgctcaattttctgttacctctagtggagtgtgaaggtaggttatggagctggggttggggagtgtcacgtctctctgggtccctgtcttttaggttccagtgtgtgttccaagtgctgccaaagtgcctcagcccagagacacccctgcctagcgtgctgctggctgttgagctcctctccctgctggcggaccacgaccagctggcacctcagctctgttcccactcaggtaaagcagggtggggcgggcatctagactgctcctgcagatcaagggaagggttggggtgggaacagggtcaagggcctcagagggcttctgcagtatgtagcccacgctctttatgagaaagggcaccctgagtgagaggtgcgctgtccttttcagaaggctgcctcctgctgctgctgtacatgtacatcacatcacggcctgacagagtggccttggagacacaatggctccagctggaacaagaggtaaaaactccagagccccttctggacactgtccccaccccatcctaagaaccaacagaatctcctttctttccgctgaggagaaacggagctttaattcattttagtaacaagagttgataaagcaactaaagaggtaaactcagtttgggggctgtgcaccacacagatgcaatgcagacaggacttggcagagttgcctgatgactgatcctgtcttggctttgggacgtgggggcagaatagggggctggaggcaggtgggttgctggtgacacatgagtgcttgtcctgggtcttccctgggaaggtcccacaccacttcttggaccccatttgtgcagacaaacagttgctgaagcaagtgaactcttatagaagcttaagaaaaccctcgcagggactggtctccttctgcccaggatggaaccactctgttcttgttctaggtggtgtggctcctggctaagcttggtgtgcagagccccttgcccccagtcactggctccaactgccagtgtaatgtggaggtgagtgggtaggggccaacagctggcagctctggtggtaagggggccccgtgcaaggactgtaggccccactgcaaaccccctcacgtgaggtggctaggctgagcggattgttagggtgcaggccatggtggcaccaggcctcagtctgcaccccccctctctcaggtggtcagagcgctcacggtgatgttgcacagacagtggctgacagtgcggagggcagggggacccccaaggaccgaccagcagaggcggacagtgcgctgtctgcgggacacggtgctgctgctgcacggcctatcgcagaaggacaagctcttcatgatgcactgcgtggaggtcctgcatcagtttgaccaggtgatgccgggggtcagcatgctcatccgagggcttcctgatgtgacggactgtgaaggtaagcctgccagaggccatcctgcccagcccccatggcttcttccagaggttccccaacaagtcagattcctgggtgtcccctcagcaggcccccgcccactgcagcctgttccagcactggggccattttcttttatcttcctgcctcagttcttctccaagcatattgggatgccttttgcatctatctgttgagtgtgcctggcagagccacggtttttccagaaatagccgtgtctgaattgcctttgtatcactttgtttgcagtagctgagggagcagggcctgggtgtggaagggactggttagttcctgcggacgttgggggaggagaggtgggacctgccctaggccctggcacctttgggccctcaccaggaacctctcctttttgtctcagaggcagccctggatgacctctgtgccgcggaaaccgatgtggaagaccccgaggtggagtgtggctgaggccctgagtgtccagccacatggtggcaccagcaccactcctttccttaccacatcaactgattaaagcagtgaccagcaggaactgcccagagaactggctggccttgtttcctgagtctgatctgtttggcggagtgggaggggtggagcaggacccggaccctgagtggctgggatccttcttcctgtccctggctgttgctgagcccgtccccatggtaactgatctgccttgaggaaggagccctgccctgcctgtggaattgtcctgagtcattgctttgggctggggccatgggaagaaaccattgtgtggcagggaaggaggtggctcttggcccaggcctaaaccaggaaagcctgggaaactgggacccacaggtgggcatgaaagggccgcagcaggggctcccagcagtgtgtaagaccgggagctggtctggcaccactgccctggtccttccagctgcctgtcactggtatgatggccccggtgcattgtgccaccagcaggccacagctgtggatcttggaaggcctctggggtcccccgggagcaggggagtgggtgtgggggggaacggatggtggtgagagggacagaccaggcaggctgacgagcagggcgggcctggctcacgtgggcctgtaggcgggcccacgccaagtttcacttcccgccactgctgccagcgagagccgcgggagagtgtgcagccgagtcactactgcctgcctgcctgcctgctacggtgagtgtggcccccacaatgggatggcgcagggcaggagggccatgggttcccccaccccagactaagggggcactaggggaggggccgagtcatgtgaagagggagaccctctcagacagtcgaatgtgctggtcccactaaggaaaccacctcaccctctccaacttcctgcctgaaaatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtaggccaagaaggaaaatctgacgaataaagacccccgctgccccatagcactgagtggtcaattggctcctacccttcttggcagcacagacctctgcccacacccagggacctggggaggagggcacagtgtttgtccctcactttccctagcaggatggtcaactatctgagcccagtaaaagtagggaaaacacagagaagcccaaatggatggaaagagtacagcctgattccaattccagcagccgaggccaggaagaaagggggcagcagctccaaacgattgcatttattataaacaagtgtacagaccctagactcagaaacacaacagatttgagctaagacagctctggtgaaacagtaatagagggaggaggaacacgaggtattcatgtctgggccagagctgccatccagggccccacaccccacctttggtccagatggcccattggcccaggtgtccctgctgccagaacaccgtggactggggtacaggagttgttgcatattccatgaggctggtgtcgggaagcagggactcacagttgccaggttgtccatctctgagccaatttccctccacaacccaggggtttcagtctcatatcaactatcatgtttgaaacagaaaacag

SEQ ID NO: 2 - protein, 314 aa, Homo sapiens
MGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 3 - DNA, 3582 bp, Homo sapiens
tgcagtctgaactccagtttaaagatgcagagatgaatgaattaaggacaaagctccagaccagtgaacgagcaaataaactggctgctccctctgtttcccatgtcagtcctaggaaaaacccttctgtggttataaagccagaagcatgttctccacaatttggaaaaacatcttttcctacaaaggagtcttttagtgctaacatgtcccttccccacccctgccagacggagtcaggatacaagcctctggtgggcagagaggatagtaagccccacagtctgagaggtgactccataaaacaagaagaggcccagaaaagctttgttgacagctggagacagagatcaaacactcaaggttccattttgataaacctgctcctgaagcagcctttgatcccagggtcatccctaagcctttgccacctcctgagtagtagttctgagtctcctgctggcacccccctgcagccaccagggtttggcagtaccttggctggaatgtcaggcctcaggaccacaggttcttatgatgggtcattttccctctcagccctgagagaagcacagaacctggcattcactggactgaatctggttgcccggaatgagtgctcacgtgatggagacccagcagagggaggcagaagggccttcccactctgccagcttcctggagccgtgcatttcctcccccttgtacagttcttcatcggcttacactgccaggccctgcaggacttggcagctgctaagagaagcggagcacctggggactcaccgacacattcctcctgcgtgagctctggggtagagaccaaccctgaggactcagtgtgcatcctggaaggcttctctgtgactgcacttagcattcttcagcacctggtgtgccacagcggagcagtcgtctccctattactgtcaggagtgggggcagattctgctgctggggaaggaaacaggagcctggttcacaggcttagtgatggagatatgacctcagccctaaggggggttgctgatgaccaaggacagcacccactgttgaagatgcttcttcacctgttggctttctcttctgcagcaacaggtcaccttcaagccagtgtcctgacccagtgccttaaggttttggtgaaattagccgaaaacacttcctgtgatttcttgcccaggttccagtgtgtgttccaagtgctgccaaagtgcctcagcccagagacacccctgcctagcgtgctgctggctgttgagctcctctccctgctggcggaccacgaccagctggcacctcagctctgttcccactcagaaggctgcctcctgctgctgctgtacatgtacatcacatcacggcctgacagagtggccttggagacacaatggctccagctggaacaagaggtggtgtggctcctggctaagcttggtgtgcagagccccttgcccccagtcactggctccaactgccagtgtaatgtggaggtggtcagagcgctcacggtgatgttgcacagacagtggctgacagtgcggagggcagggggacccccaaggaccgaccagcagaggcggacagtgcgctgtctgcgggacacggtgctgctgctgcacggcctatcgcagaaggacaagctcttcatgatgcactgcgtggaggtcctgcatcagtttgaccaggtgatgccgggggtcagcatgctcatccgagggcttcctgatgtgacggactgtgaagaggcagccctggatgacctctgtgccgcggaaaccgatgtggaagaccccgaggtggagtgtggctgaggccctgagtgtccagccacatggtggcaccagcaccactcctttccttaccacatcaactgattaaagcagtgaccagcaggaactgcccagagaactggctggccttgtttcctgagtctgatctgtttggcggagtgggaggggtggagcaggacccggaccctgagtggctgggatccttcttcctgtccctggctgttgctgagcccgtccccatggtaactgatctgccttgaggaaggagccctgccctgcctgtggaattgtcctgagtcattgctttgggctggggccatgggaagaaaccattgtgtggcagggaaggaggtggctcttggcccaggcctaaaccaggaaagcctgggaaactgggacccacaggtgggcatgaaagggccgcagcaggggctcccagcagtgtgtaagaccgggagctggtctggcaccactgccctggtccttccagctgcctgtcactggtatgatggccccggtgcattgtgccaccagcaggccacagctgtggatcttggaaggcctctggggtcccccgggagcaggggagtgggtgtgggggggaacggatggtggtgagagggacagaccaggcaggctgacgagcagggcgggcctggctcacgtgggcctgtaggcgggcccacgccaagtttcacttcccgccactgctgccagcgagagccgcgggagagtgtgcagccgagtcactactgcctgcctgcctgcctgctacggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtaggccaagaaggaaaatctgacgaataaagacccccgctgcccc

SEQ ID NO: 4 - DNA, 20 bp, Homo sapiens
caattctaggcttcccctcc

SEQ ID NO: 5 - DNA, 20 bp, Homo sapiens
agatgtgagcggacataccc

SEQ ID NO: 6 - DNA, 20 bp, Homo sapiens
agtagttttgtaactgcggg

SEQ ID NO: 7 - DNA, 20 bp, Homo sapiens
tacataccccagtgtcggag

SEQ ID NO: 8 - DNA, 20 bp, Homo sapiens
ctgacgggtatgtccgctca

SEQ ID NO: 9 - DNA, 20 bp, Homo sapiens
acaaacagggagtgaaaggg

(19) United States
(12) Patent Application Publication
(10) Pub. No.: US 2010/0104579 A1
(43) Pub. Date: Apr. 29, 2010

(54) TREX1 AS A MARKER FOR LUPUS ERYTHEMATOSUS

(75) Inventors: Norbert Hubner, Berlin (DE); Min Ae. Lee-Kirsch, Dresden (DE)

Correspondence Address: DAVID S. RESNICK, NIXON PEABODY LLP, 100 SUMMER STREET, BOSTON, MA, 02110-2131, US

(73) Assignee: TECHNISCHE UNIVERSITAT DRESDEN, Dresden (DE)

(21) Appl. No.: 12/442,762

(22) PCT Filed: Jul. 19, 2007
(86) PCT No.: PCT/EP07/06437

§ 371 Date: Dec. 8, 2009

(30) Foreign Application Priority Data
Sep. 25, 2006 (EP) 06020014.4

(51) Int. Cl. A61K 39/395; C12Q 1/68; C12Q 1/44; A61K 31/7052; A61K 38/16; A61P 37/00

(52) U.S. Cl. 424/158.1; 435/6; 435/19; 514/44 R; 514/12

(57) ABSTRACT

The present invention relates a method of diagnosing lupus erythematosus or related autoimmune diseases in a human, said method comprising at least one of detecting a genetic variant, a mutation or polymorphism in the gene encoding for TREX1, detecting a change in the biochemical activity of TREX1, and detecting a change in the expression of the gene encoding for TREX1. The present invention further relates to the use of TREX1 for therapy and as a target for new therapeutic approaches.

The present invention relates a method of diagnosing lupus erythematosus in a human, said method comprising at least one of detecting a genetic variant (mutation or polymorphism) in the gene encoding for TREX1, detecting a change in the biochemical activity of TREX1, and detecting a change in the expression of the gene encoding for TREX1. The present invention further relates to the use of TREX1 for therapy of lupus erythematosus and related autoimmune diseases and as a target for new therapeutical approaches.

Systemic lupus erythematosus is a complex autoimmune disease with a prevalence of 0.06% in the general population. Its etiology is multifactorial and influenced by both genetic and environmental factors (Alarcon-Riquelme, M. E. (2005) The genetics of systemic lupus erythematosus. J. Autoimmun. 25 Suppl, 46-48; Cunninghame Graham, D. S., Vyse, T. J. (2004) The candidate gene approach: have murine models informed the study of human SLE? Clin Exp. Immunol. 137, 1-7). The clinical picture is characterized by a great variety of symptoms, ranging from mild to lethal. The disease can affect almost any organ in the body, causing arthritis, vasculitis, thrombosis, anemia, heart disease, or kidney failure. Cutaneous findings are a hallmark of the disease and include butterfly rash, discoid lesions, oral ulcers, and alopecia (Sontheimer, R. D. (1997) The lexicon of cutaneous lupus erythematosus—a review and personal perspective on the nomenclature and classification of the cutaneous manifestations of lupus erythematosus. Lupus 6, 84-95). Symptoms usually recur over time, and may also include psychosis and cognitive decline. To date the diagnosis rests on clinical and laboratory criteria defined by the American Rheumatic Association. Thus, the diagnosis is made if 4 of the 11 diagnostic criteria for systemic lupus erythematosus can be found in an affected individual (Mills, J. A. (1994) Systemic lupus erythematosus. N. Engl. J. Med. 330, 1871-1879). However, the insidious onset of the disease and the variable clinical picture at the time of manifestation remain a diagnostic challenge even for the specialist and in many cases the diagnosis is made only when irreparable damage such as renal failure has occurred. The most difficult aspect with respect to management of patients with lupus is to treat the active phase without allowing the treatment to cause long-term damage. Nonsteroidal antiinflammatory drugs remain the mainstay of treatment in patients with polyarthralgias or mild polyarthritis, while for cutaneous manifestations the strict use of sunblock along with judicious use of topical steroids is the treatment of choice. Many patients with systemic or cutaneous lupus erythematosus, however, will need systemic glucocorticoids, antimalarials such as hydroxychloroquine or the more opthalmotoxic chloroquine, and immunosuppressive agents such as methotrexate, cyclophoaphamide, or azathioprine at some point during the course of the illness.

The formation of immune complexes consisting of autoantibodies against nuclear antigens is thought to be the principal cause of the inflammatory process leading to skins rashes, vasculitis, arthritis, and nephritis (Mills, J. A. (1994) Systemic lupus erythematosus. N. Engl. J. Med. 330, 1871-1879; Walport, M. J. (2000) Lupus, DNase and defective disposal of cellular debris. Nat. Genet. 25, 135-136). To date, several susceptibility loci have been identified by both genome-wide and association approaches (Alarcon-Riquelme, M. E. (2005) The genetics of systemic lupus erythematosus. J. Autoimmun. 25 Suppl, 46-48; Cunninghame Graham, D. S., Vyse, T. J. (2004) The candidate gene approach: have murine models informed the study of human SLE? Clin Exp. Immunol. 137, 1-7). However, most of the genetic basis and the molecular pathogenesis of lupus erythematosus remain undefined.

Apart from autosomal recessively inherited deficiencies of complement factors, which play an important role in adaptive immunity, no monogenic form of lupus erythematosus has been identified so far. Thus, selective deficiency of C1q, C1r, C1s, C2, or C4a has been associated with multiple autoimmune diseases including a lupus-like phenotype (Lee, S. L., Wallace, S. L., Barone, R., Blum, L., Chase, P. H. (1978) Familial deficiency of two subunits of the first component of complement. C1 r and C1 s associated with a lupus erythematosus-like disease. Arthritis Rheum. 21, 958-967; Dragon-Durey, M. A., Quartier, P., Fremeaux-Bacchi, V., Blouin, J., de Barace, C., Prieur, A. M., Weiss, L., Fridman, W. H. (2001) Molecular basis of a selective C1s deficiency associated with early onset multiple autoimmune diseases. J. Immunol. 166, 7612-7616; Hannema, A. J., Kluin-Nelemans, J. C., Hack, C. E., Eerenberg-Belmer, A. J., Mallee, C., van Helden, H. P. (1984) SLE like syndrome and functional deficiency of C1q in members of a large family. Clin Exp. Immunol. 55, 106-114; Provost, T. T., Arnett, F. C., Reichlin, M. (1983) Homozygous C2 deficiency, lupus erythematosus, and anti-Ro (SSA) antibodies. Arthritis Rheum. 26, 1279-1282), while selective deficiency of C3 and C5 leads to high susceptibility to bacterial infections in addition to lupus-like phenotypes (Alper, C. A., Bloch, K. J., Rosen, F. S. (1973) Increased susceptibility to infection in a patient with type II essential hypercatabolism of C3. N. Engl. J. Med. 288, 601-606; Snyderman, R., Durack, D. T., McCarty, G. A., Ward, F. E., Meadows, L. (1979) Deficiency of the fifth component of complement in human subjects. Clinical, genetic and immunologic studies in a large kindred. Am. J. Med. 67, 638-645).

Genes that have been implicated in the pathogenesis of lupus erythematosus or other autoimmune diseases are for example genes encoding toll like receptor 9, [TLR9, MIM *605474] (Ng, M. W., Lau, C. S., Chan, T. M., Wong, W. H., Lau, Y. L. (2005) Polymorphisms of the toll-like receptor 9 (TLR9) gene with systemic lupus erythematosus in Chinese. Rheumatology. (Oxford) 44, 1456-1457; Barrat, F. J., Meeker, T., Gregorio, J., Chan, J. H., Uematsu, S., Akira, S., Chang, B., Duramad, O., Coffman, R. L. (2005) Nucleic acids of mammalian origin can act as endogenous ligands for Toll-like receptors and may promote systemic lupus erythematosus. J. Exp. Med. 202, 1131-1139), deoxyribonuclease I-like 3 [DNASE1L3, MIM *602244] (Walport, M. J. (2000) Lupus, DNase and defective disposal of cellular debris. Nat. Genet. 25, 135-136, Wilber, A., O'Connor, T. P., Lu, M. L., Karimi, A., Schneider, M. C. (2003) Dnase113 deficiency in lupus-prone MRL and NZB/W F1 mice. Clin Exp. Immunol. 134, 46-52), ubiquitin-activating enzyme E1-like [UBE1 L, MIM *191325] (Vinuesa, C. G., Cook, M. C., Angelucci, C., Athanasopoulos, V., Rui, L., Hill, K. M., Yu, D., Domaschenz, H., Whittle, B., Lambe, T., Roberts, I. S., Copley, R. R., Bell, J. I., Cornall, R. J., Goodnow, C. C. (2005) A RING-type ubiquitin ligase family member required to repress follicular helper T cells and autoimmunity. Nature 435, 452-458), protein kinase C delta [PRKCD, MIM *176977] (Mecklenbrauker, I., Saijo, K., Zheng, N.Y., Leitges, M., Tarakhovsky, A. (2002) Protein kinase Cdelta controls self-antigen-induced B-cell tolerance. Nature 416, 860-865; Miyamoto, A., Nakayama, K., Imaki, H., Hirose, S., Jiang, Y., Abe, M., Tsukiyama, T., Nagahama, H., Ohno, S., Hatakeyama, S., Nakayama, K. I. (2002) Increased proliferation of B cells and auto-immunity in mice lacking protein kinase Cdelta. Nature 416, 865-869), interleukin 17 receptor B and D [IL17RB, MIM *605458; IL17RD, MIM *606807] (Kurasawa, K., Hirose, K., Sano, H., Endo, H., Shinkai, H., Nawata, Y., Takabayashi, K., Iwamoto, I. (2000) Increased interleukin-17 production in patients with systemic sclerosis. Arthritis Rheum. 43, 2455-2463), and the chemokine receptor cluster [CCR9, MIM *604738; CXCR6, MIM *605163; XCR1, MIM *600552; CCR1, MIM *601159; CCR2, MIM *601267; CCR3, MIM *601268; CCR5, MIM *601373; CCRL2, MIM *608379) (Amoura, Z., Combadiere, C., Faure, S., Parizot, C., Miyara, M., Raphael, D., Ghillani, P., Debre, P., Piette, J. C., Gorochov, G. (2003) Roles of CCR2 and CXCR3 in the T cell-mediated response occurring during lupus flares. Arthritis Rheum. 48, 3487-3496; Eriksson, C., Eneslatt, K., Ivanoff, J., Rantapaa-Dahlqvist, S., Sundqvist, K. G. (2003) Abnormal expression of chemokine receptors on T-cells from patients with systemic lupus erythematosus. Lupus 12, 766-774).

Prokunina et al. (in Prokunina L, et al. A regulatory polymorphism in PDCD1 is associated with susceptibility to systemic lupus erythematosus in humans Nat. Genet. 2002 December; 32(4):666-9. Epub 2002 Oct. 28.) describe a study for common polymorphism of the PDCD1 gene that was found to be associated with systemic lupus erythematosus (SLE) using an association study design. In contrast to the approach described in Prokunina et al., the present invention uses a resequencing approach and identifies rare pathogenic variants of the TREX1 gene in patients with common complex forms of SLE. In addition, the inventors identify a mutation in TREX1 in a monogenic autosomal dominant form of lupus. Thus, these variants represent disease causing mutations. In fact, when using the approach by Prokunina et al., the inventors would not have found any evidence for an association of a common variant of the TREX1 gene with SLE.

WO 2005/098045 describes a diagnostic method for the detection of SLE in a human body sample through identification of Mycoplasma haemosapiens.

U.S. Pat. No. 6,632,665 describes the isolated nucleic acid molecule encoding the genomic sequence encoding the human 3′-5′ exonuclease TREX1. Methods of use include inhibition of exonuclease activity to increase incorporation of nucleotide analogs into DNA in rapidly dividing cells.

WO 2005/051988 broadly describes compositions containing 596 proteins and methods of using those compositions for the diagnosis and treatment of immune related diseases, such as systemic lupus erythematosus. Sequence 200 from WO 2005/051988 encodes for a part of the protein TREX1. WO 2005/051988 focuses on expression analysis, but does not examine mutations or polymorphisms in said genes.

JP 2006-101712 describes a method for diagnosing the systemic lupus erythematosus by analyzing a nucleic acid molecule group containing each of the nucleotide sequences of a plurality of genes selected from the groups of genes specifically expressed in cells presenting in the peripheral blood of a subject having the systemic lupus erythematosus.

Despite the above attempts, there is an ongoing need for the identification of genes responsible for lupus, which may shed light onto the pathogenesis of common forms of connective tissue diseases such as systemic lupus erythematosus, and will allow the development of new diagnostic and therapeutic approaches. Alternatively, also markers for monogenic forms of lupus erythematosus are sought for. In addition, recognition of disease causing genetic variants may also help to define subgroups of patients based on the genetic etiology and may allow prediction of the clinical course and the response to specific therapeutic measures.

In order to solve this problem, according to a first preferred aspect thereof, the present invention provides a method of diagnosing lupus erythematosus in a human, said method comprising at least one of detecting a genetic variant (mutation or polymorphism) in the gene encoding for TREX1, detecting a change in the biochemical activity of TREX1, and detecting a change in the expression of the gene encoding for TREX1. The test can be easily performed at the onset of symptoms even if the clinical picture does not fulfill the diagnostic criteria defined by the American Rheumatic Association. Thus, this test could speed up diagnosis and institution of specific therapy.

Lupus erythematosus, and in particular systemic lupus erythematosus (SLE) is a complex polygenic disease and considered a prototypic autoimmune disease. The locus identified in the present invention does not localize within any hitherto mapped susceptibility locus for systemic lupus erythematosus (Alarcon-Riquelme, M. E. (2005) The genetics of systemic lupus erythematosus. J. Autoimmun. 25 Suppl, 46-48; Cunninghame Graham, D. S., Vyse, T. J. (2004) The candidate gene approach: have murine models informed the study of human SLE? Clin Exp. Immunol. 137, 1-7), previous studies of complex diseases have demonstrated that genome-wide and association approaches may lead to completely different results.

The gene trex1 encodes for a DNase III-enzyme and maps to chromosome 3p21.3-p21.2. The protein was falsely used synonymously with ATRIP (ATR-interacting protein), nevertheless, ATRIP-ORF is encoded by 5′ prime exons of TREX1 gene in a different reading frame. Trex1 includes 11 exons, wherein the coding complete region is localized in exon 11. TREX1 consist of 314 AA, as shown in SEQ ID No. 2. TREX1 appears to be ubiquitously expressed in the human, however some data suggest high expression in lymphocytes. Yet, no role of TREX1 in lupus erythematosus has been described, in contrast, TREX−/− mice die of autoimmune cardiomyocarditis (Morita et al. (2004) Gene-targeted mice lacking the Trex1 (DNase III) 3′-5′DNA exonuclease develop inflammatory myocarditis. Mol. Cell. Biol. 24, 6719-6727).

In yet another embodiment, the present invention concerns a method of diagnosing lupus erythematosus in a human, wherein said diagnosis further includes the step of an analysis of the risk to develop a lupus erythematosus or an analysis of the risk for a relapse of lupus erythematosus in said human.

In yet another embodiment, the present invention concerns a method of diagnosing lupus erythematosus in a human according to the present invention, wherein detection of said change of expression comprises a quantitative or qualitative detection method. Such methods are well known and comprise methods that quantify the amount of TREX1-protein or of TREX1-encoding mRNA. Preferred is a method of diagnosing lupus erythematosus in a human according to the present invention, wherein detection of said change of expression comprises using a TREX1-specific antibody or a trex 1-mRNA specific RT-PCR. Thus, the detection may be qualitative or quantitative, and may be performed in comparison with monitoring a complex formation in a control sample of known normal tissue cells of the same cell type. A larger quantity of complexes formed in the test sample indicates the presence or absence of lupus erythematosus in the human from which the test tissue cells were obtained. The TREX1-specific antibody preferably carries a detectable label. Complex formation can be monitored, for example, by light microscopy, flow cytometry, fluorimetry, or other techniques known in the art. The test sample is usually obtained from an individual suspected of having lupus erythematosus.

In another embodiment, the invention provides a method for determining the presence of TREX1 polypeptide in a sample obtained from an individual suspected of having lupus erythematosus comprising exposing a test sample of cells suspected of containing the TREX1-polypeptide to an anti-TREX1 antibody, and determining the binding of said antibody to said cell sample. In a specific aspect, the sample comprises a cell suspected of containing the TREX1-polypeptide and the antibody binds to the cell. The antibody is preferably detectably labeled and/or bound to a solid support. The present invention further provides said specific anti-TREX1 antibodies. Exemplary antibodies include polyclonal, monoclonal, humanized, bispecific, and heteroconjugate antibodies.

In another embodiment, the invention provides a method of diagnosing SLE in a mammal which comprises detecting the presence or absence of the TREX1 polypeptide in a test sample of tissue cells obtained from said mammal, wherein the presence or absence of the TREX1 polypeptide in said test sample is indicative of the presence of SLE in said mammal.

Most preferred is the use of a TREX1-specific antibodies in order to distinguish between mutated and non-mutated TREX1-polypeptides, as described herein in the Tables below, or in order to detect quantitative tissues-specific changes in protein expression.

In yet another embodiment, the present invention concerns a method of diagnosing lupus erythematosus in a human according to the present invention, wherein detection of said change of biochemical activity comprises a 3′ nucleotide excision assay. Examples of such assays are known in the literature and described, for example, in Mazur et al. (Mazur D J, Perrino F W. Excision of 3′ termini by the Trex1 and TREX2 3′-->5′ exonucleases. Characterization of the recombinant proteins. J Biol. Chem. 2001 May 18; 276(20):17022-9. Epub 2001 Mar. 6.).

A particularly preferred embodiment of the present invention then relates to a method of diagnosing lupus erythematosus in a human according to the present invention, wherein said method comprises the steps of: a) obtaining a biological test sample from a human to be diagnosed, preferably a human suspected of having or being predisposed to lupus erythematosus, b) analyzing the sequence of the gene encoding for TREX1 in said sample, c) comparing said sequence as analyzed with a sequence of the gene encoding for TREX1 in a control sample from a healthy individual, and d) diagnosing lupus erythematosus in said human to be diagnosed based on the differences between the two samples, wherein the presence of a genetic variant (mutation or polymorphism) in the test sample as compared to the control sample is indicative of the risk and/or presence of lupus erythematosus.

For example, antibodies, including antibody fragments, can be used to qualitatively or quantitatively detect the expression of TREX1 protein (“marker gene product”). The antibody preferably is equipped with a detectable, e.g., fluorescent label, and binding can be monitored by light microscopy, flow cytometry, fluorimetry, or other techniques known in the art. It is speculated by the inventors that the presence of a mutation or mutations in the gene encoding for TREX1 will have an effect in that the activity and/or presence of TREX1 is reduced. Since TREX1 is regarded as playing an important role in the degradation/control of single-stranded DNA in the nucleus of the cell, it is likely that a malfunction of TREX1 will lead to an increased level of single-stranded DNA which constitutes an (auto)immune target and might be one factor for the development of the autoimmune diseases as described herein.

In situ detection of antibody binding to the TREX1 gene product can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

Preferred is a method of diagnosing lupus erythematosus in a human according to the present invention, wherein analyzing the sequence of the gene encoding for TREX1 comprises the detection of genetic variants (mutations and polymorphisms) in the regulatory regions, introns and/or exons, in particular within the coding exon of the trex1-gene. Thus, the term “gene encoding for TREX1” in the context of the present invention encompasses the coding as well as the non-coding regions of the gene, together with any regulatory regions that are responsible for a control of TREX1-expression, such as promoter and enhancer regions, as well as 5′- and/or 3′-non-coding regions of trex1. Most preferred is an analysis of the region as depicted in SEQ ID No. 1 or 3.

Mutations are differences in the nucleic acid sequence of a gene that change the encoded protein or its expression thereby changing its function and/or its transcriptional regulation.

Polymorphisms are differences in the nucleic acid sequence of a gene and are differences in DNA sequence among individuals. Genetic variations occurring in more than 1% of a population would be considered useful polymorphisms for the present analysis. Therefore, all methods for analyzing nucleic acid sequences can be used in the context of the present invention. Preferred is a method of diagnosing lupus erythematosus in a human according to the present invention, wherein detection of said genetic variants (mutations or polymorphisms) comprises at least one of an analysis comprising hybridization, restriction enzyme analysis, and sequencing.

A particularly preferred embodiment of the present invention then relates to a method of diagnosing lupus erythematosus in a human according to the present invention, wherein said genetic variants (mutations and polymorphisms) to be detected are selected from the group of genetic variants as listed in Table 1 and/or 2. Preferably, 1, 2, 3, 4, 5 or more mutations can be analyzed.

Table 1 below shows the results of the first sequence analysis of all exons including flanking intronic regions of the Trex 1 gene among 90 unrelated patients with SLE and 96 unrelated healthy controls. Several genetic variants that were present only among individuals affected with SLE and not among healthy controls could be identified.

TABLE 1
Polymorphisms in the TREX1 gene I

| Position and name of | Number of | Number of | |
|---|---|---|---|
| polymorphism according | alleles found | alleles found | |
| to genomic sequence | in SLE cases | in controls | |
| (SEQ ID No. 1) | (n = 90) | (n = 96) | Description |
| g.3829A > T | 1 (T-allele) | 0 | Synonymous¹ |
| g.3858A > G | 3 (G-allele) | 5 | Synonymous¹ |
| g.4032A > C | 1 (C-allele) | 0 | Synonymous¹ |
| rs11925638 | | | |
| g.11170C > T | 1 (T-allele) | 0 | Synonymous¹ |
| rs35240314 | | | |
| g.14283G > A | 1 (A-allele) | 0 | Synonymous¹ |
| g.14384C > T | 1 (T-allele) | 0 | Synonymous¹ |
| g.14455T > C | 1 (C-allele) | 0 | Synonymous¹ |
| g.17696C > T | 1 (T-allele) | 0 | Synonymous¹ |
| rs34426134 | | | |
| g.18682C > A | 2 (A-allele) | 0 | Synonymous¹ |
| rs3135936 | | | |
| g.18743G > A | 2 (A-allele) | 4 | Synonymous¹ |
| g.118853A > T | 1 (T-allele) | 0 | Synonymous¹ |
| g.20131T > C | 42 (T-allele) | 26 | Synonymous¹ |
| rs3135941 | | | |
| g.20570G > A | 1 (A-allele) | 0 | D18N² |
| g.20852C > A | 1 (A-allele) | 1 | L112M |
| g.21049C > T | 73 (T-allele) | 69 | Synonymous¹ |
| rs11797 | | | |
| g.21197G > A | 1 (A-allele) | 0 | G227S |
| g.21238G > C | 1 (C-allele) | 0 | R240S |
| g.21257G > C | 1 (C-allele( | 0 | A247P |
| g.21315A > G | 1 (G-allele) | 0 | E266G |
| g.21330\hat{ } 21331insAA | 1 (insAA) | 0 | K271fs272R |
| g.21430G > A | 4 (A-allele) | 0 | Synonymous¹ |
| rs3135945 | | | |
| g.21500T > C | 3 (C-allele) | 0 | 3′-untranslated |
| rs3135946 | | | region |

¹with reference to Trex1 open reading frame.
²mutation in family with monogenic chilblain lupus.

Lupus erythematosus usually is a multifactorial disease, i.e. several genes are involved in the development and/or the predisposition of said disease. The present invention now provides a novel marker for lupus erythematosus, the gene for TREX1, which can either be used as an additional marker in combination with existing lupus-markers (see above), or even as a mono-marker for the presence of lupus erythematosus.

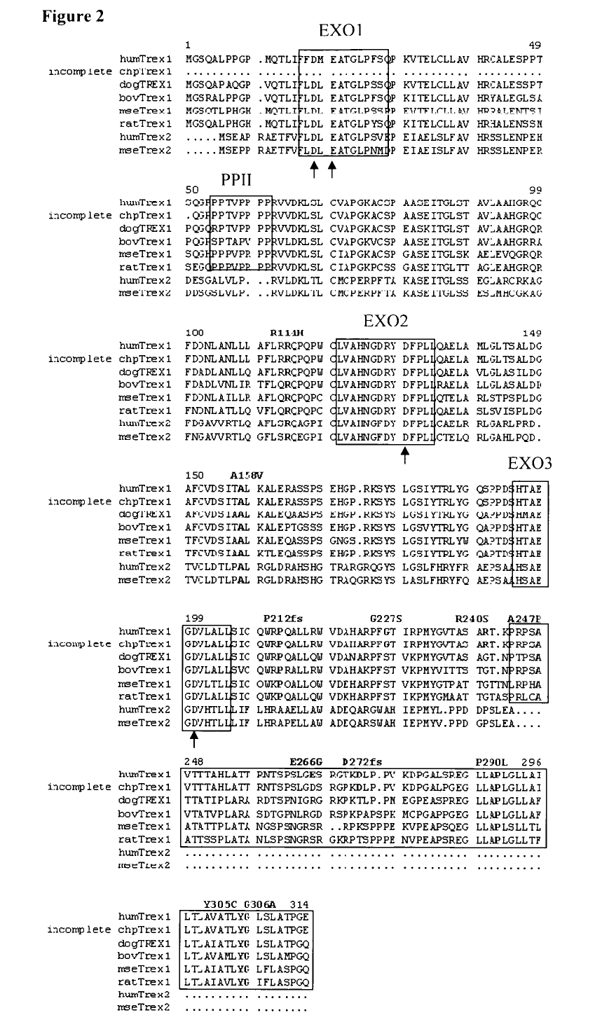

In a preferred method of diagnosing lupus erythematosus in a human according to the present invention, analyzing the sequence of the gene encoding for TREX1 comprises the detection of genetic variants (mutations or polymorphisms) in the C-terminal 150, preferably 100, amino acids of the human TREX1 protein (according to FIG. 2), and/or in at least one conserved region of the gene encoding for TREX1, such as in position 114, 158, 212, 227, 240, 247, 272, 290, 305 and/or 306 of the human TREX1 protein and directly adjacent amino acid positions, or the homologous positions thereto in a TREX1 protein from another species, such as monkey or dog or mouse, or even human TREX2 protein. A “conserved region” is a region of the gene, wherein the amino acid sequence as encoded exhibits at least 60%, preferably 80%, more preferably 90%, conserved (preferably identical) TREX1-amino acids between the species as compared, such as, for example, in FIG. 2, where six species are compared. Preferred are four (e.g. human, mouse, rat, and monkey). Examples for conserved regions according to FIG. 2 are therefore the regions of position 114 (all conserved), 158 (all conserved), 212 (all conserved), 227, 240, 247 (all conserved), 272, 290 (all conserved), 305 (all conserved) and/or 306 (all conserved) of the human TREX1 protein and directly adjacent amino acid positions.

In a preferred method of diagnosing lupus erythematosus in a human according to the present invention, said (genetic) markers are variants that are selected from the group of polymorphisms g.3829A>T; g.3858A>G; g.4032A>C, rs11925638; g.11170C>T, rs35240314; g.14283G>A; g.14384C>T; g.14455T>C; g.17696C>T, rs34426134; g.18682C>A, rs3135936; g.18743G>A; g.18853A>T; g.20131T>C, rs3135941; g.20570G>A; g.20852C>A; ss71651743, 20701 G>A; ss71651744, 20707 G>A; rs3135943, 20716 G>A; ss71651745, 20830 G>C; ss71651746, 20857 G>A; ss71651747, 20914 C>T; rs3135944, 20980 T>C; ss71651748, 21004 G>A; g.21049C>T, rs11797; g.21197G>A; ss71651749, 21208 G>A; g.21238G>C; g.21257G>C; g.21315A>G; g.21330̂ 21331insAA; g.21430G>A, rs3135945; g.21500T>C, rs3135946; and ss71651751, 21521 C>T.

Another aspect of the present invention therefore relates to a method of diagnosing lupus erythematosus in a human according to the present invention, wherein said lupus erythematosus is selected from systemic lupus erythematosus, subacute cutaneous lupus erythematosus, cutaneous lupus erythematosus including chilblain lupus erythematosus, rheumatoid arthritis, Sjögren's syndrome, autoimmune nephritis, autoimmune vasculitis, autoimmune hepatitis, autoimmune carditis, autoimmune encephalitis, autoimmune mediated hematological disease, and other autoimmune diseases.

A particularly preferred embodiment of the present invention then relates to a method of diagnosing lupus erythematosus in a human according to the present invention, which further comprising the analysis of other genetic markers for lupus erythematosus, such as MICA, CTLA4, CD154 (3′UTR), PDCD1, RNUX1, PTP22, SCL22A4, SUMO4, and IRF5). Additional markers are known from the literature (see, for example, Roberton C A, Vyse T J. The genetics of systemic lupus erythematosus. Exp Nephrol. 2000 July-October; 8(4-5):194-202.; Gambelunghe G, Gerli R, Bocci E B, Del Sindaco P, Ghaderi M, Sanjeevi C B, Bistoni O, Bini V, Falorni A. Contribution of MHC class I chain-related A (MICA) gene polymorphism to genetic susceptibility for systemic lupus erythematosus. Rheumatology (Oxford). 2005 March; 44(3):287-92. Epub 2004 Nov. 2. Torres B, Aguilar F, Franco E, Sanchez E, Sanchez-Roman J, Jimenez Alonso J, Nunez-Roldan A. Martin J, Gonzalez-Escribano M F. Association of the CT60 marker of the CTLA4 gene with systemic lupus erythematosus. Arthritis Rheum. 2004 July; 50(7):2211-5.; Citores M J, Rua-Figueroa I, Rodriguez-Gallego C, Durantez A, Garcia-Laorden M I, Rodriguez-Lozano C, Rodriguez-Perez J C, Vargas J A, Perez-Aciego P. The dinucleotide repeat polymorphism in the 3′UTR of the CD 154 gene has a functional role on protein expression and is associated with systemic lupus erythematosus. Arm Rheum Dis. 2004 March; 63(3):310-7.; Tokuhiro S, Yamada R, Chang X, Suzuki A, Kochi Y, Sawada T, Suzuki M, Nagasaki M, Ohtsuki M, Ono M, Furukawa H, Nagashima M, Yoshino S, Mabuchi A, Sekine A, Saito S, Takahashi A, Tsunoda T, Nakamura Y, Yamamoto K. An intronic SNP in a RUNX1 binding site of SLC22A4, encoding an organic cation transporter, is associated with rheumatoid arthritis. Nat. Genet. 2003 December; 35(4):341-8. Epub 2003 Nov. 9., Reddy M V, Johansson M, Sturfelt G, Jonsen A, Gunnarsson I, Svenungsson E, Rantapaa-Dahlqvist S, Alarcon-Riquelme M E. The R620W C/T polymorphism of the gene PTPN22 is associated with SLE independently of the association of PDCD1. Genes Immun. 2005 December; 6(8):658-62, Prokunina L, Castillejo-Lopez C, Oberg F, Gunnarsson I, Berg L, Magnusson V, Brookes A J, Tentler D, Kristjansdottir H, Grondal G, Bolstad A I, Svenungsson E, Lundberg I, Sturfelt G, Jonssen A, Truedsson L, Lima G, Alcocer-Varela J, Jonsson R, Gyllensten U B, Harley J B, Alarcon-Segovia D, Steinsson K, Alarcon-Riquelme M E. A regulatory polymorphism in PDCD1 is associated with susceptibility to systemic lupus erythematosus in humans. Nat. Genet. 2002 December; 32(4):666-9. Epub 2002 Oct. 28. Orozco G, Sanchez E, Gomez L M, Gonzalez-Gay M A, Lopez-Nevot M A, Tones B, Ortego-Centeno N, Jimenez-Alonso J, de Ramon E, Sanchez Roman J, Anaya J M, Sturfelt G, Gunnarsson I, Svennungsson E, Alarcon-Riquelme M, Gonzalez-Escribano M F, Martin J. Study of the role of functional variants of SLC22A4, RUNX1 and SUMO4 in systemic lupus erythematosus. Ann Rheum Dis. 2006 June; 65(6):791-5. Epub 2005 Oct. 25., Graham R R, Kozyrev S V, Baechler E C, Reddy M V, Plenge R M, Bauer J W, Ortmann W A, Koeuth T, Gonzalez Escribano M F; Argentine and Spanish Collaborative Groups; Pons-Estel B, Petri M, Daly M, Gregersen P K, Martin J, Altshuler D, Behrens T W, Alarcon-Riquelme M E. A common haplotype of interferon regulatory factor 5 (IRF5) regulates splicing and expression and is associated with increased risk of systemic lupus erythematosus. Nat. Genet. 2006 May; 38(5):550-5. Epub 2006 Apr. 16).

The marker of the present invention can be used by the attending physician in order to apply a preventive treatment to a human in order to ameliorate a relapse or even avoid onset of the autoimmune disease (as discussed in for example. Harel M, Shoenfeld Y. Predicting and preventing autoimmunity, myth or reality? Ann N Y Acad. Sci. 2006 June; 1069:322-45.). Another aspect of the method of diagnosing lupus erythematosus in a human according to the present invention, therefore further includes the step of selecting measures for secondary or primary prevention based on said diagnosis. Such measures could be the prevention of antibodies that are accrued by patients throughout a foreseen course during the years prior to the clinical symptoms. Another aspect of the present invention relates to a classification of the lupus erythematosus in order to provide a more effective therapy based on a precise diagnosis of the pathological condition and the individual needs of the patient (“personalized treatment”), which can be readily performed by the attending physician based on the information as provided herein as well as the respective literature (see, for example, Centola M, Frank M B, Bolstad A I, Alex P, Szanto A, Zeher M, Hjelmervik T O, Jonsson R, Nakken B, Szegedi G, Szodoray P. Genome-scale assessment of molecular pathology in systemic autoimmune diseases using microarray technology: a potential breakthrough diagnostic and individualized therapy-design tool. Scand J. Immunol. 2006 September; 64(3):236-42.; and references as cited therein).

A particularly preferred embodiment of the present invention then relates to a method for identifying a compound which modifies the activity of TREX1, said method comprising contacting cells expressing TREX1 with a candidate compound, and identifying said compound by determining the activity of TREX1 in response to said candidate compound. Preferred is a method of identifying a compound which modifies the activity of TREX1 according to the present invention, wherein the activity of TREX1 is selected from the biochemical activity or the expression of the gene encoding for TREX1. The compound can be an agonist or antagonist of the TREX1 activity.

The term “antagonist” is used in the broadest sense, and includes any molecule that partially or fully blocks, inhibits, or neutralizes the biological activity of a native TREX1 polypeptide disclosed herein. In a similar manner, the term “agonist” is used in the broadest sense and includes any molecule that mimics a biological activity of a native TREX1 polypeptide disclosed herein. Suitable agonist or antagonist molecules specifically include agonist or antagonist antibodies or antibody fragments, fragments or amino acid sequence variants of native TREX1 polypeptides, peptides, antisense oligonucleotides, small organic molecules, etc. Methods for identifying agonists or antagonists of a TREX1 polypeptide may comprise contacting a TREX1 polypeptide with a candidate agonist or antagonist molecule and measuring a detectable change in one or more biological activities normally associated with the TREX1 polypeptide, such as described herein.

Preferred is a method for identifying a compound that modifies the activity of TREX1 according to the present invention, wherein said determining of said change of expression comprises a TREX1 antibody and/or an RT-PCR and/or a 3′ nucleotide excision assay, as described above for the diagnosis. Further preferred is a method for identifying a compound that modifies the activity of TREX1 according to the present invention, wherein said candidate compound is a TREX1 antisense nucleic acid or a TREX1-derived polypeptide, which is optionally mutated and/or chemically modified. Preferred is a TREX1-peptide that is mutated in order to function as a competitor for the TREX1 biological function in the cell.

In yet another embodiment, the present invention concerns a diagnostic kit for lupus erythematosus, comprising: a container comprising materials for performing a diagnostic method according to the present invention, together with suitable carriers and instructions for use.

In another embodiment, the present invention concerns an lupus erythematosus-related disease diagnostic kit, comprising an anti-TREX1 antibody and a carrier in suitable packaging. The kit preferably contains instructions for using the antibody to detect the presence of the TREX1 polypeptide, in particular mutated TREX1 polypeptide as described herein. Preferably, the carrier is pharmaceutically acceptable.

In another embodiment of the invention, a kit useful for the diagnosis or treatment of the disorders described herein is provided. The kit comprises a container and an instruction. Suitable containers include, for example, bottles, vials, syringes, and test tubes. The containers may be formed from a variety of materials such as glass or plastic. The container holds a composition which is effective for diagnosing or treating lupus erythematosus, and may have a sterile access port (for example the container may be an intravenous solution bag or a vial having a stopper pierceable by a hypodermic injection needle). The active agent in the composition is usually a polypeptide, a compound or an antibody of the invention. An instruction or label on, or associated with, the container indicates that the composition is used for diagnosing or treating the lupus-condition of choice. The kit may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Compounds identified by the above described screening assays can be formulated into a pharmaceutical composition, using standard techniques well known in the art.

In yet another embodiment, the invention provides a pharmaceutical preparation, comprising a compound as identified according to the present invention, and/or an active agent according to the present invention, together with a pharmaceutically acceptable carrier and/or excipient.

In another embodiment, the invention relates to the use of a compound as identified according to the present invention, or a pharmaceutical preparation according to the present invention for the production of a medicament for the treatment of systemic lupus erythematosus, subacute cutaneous lupus erythematosus, cutaneous lupus erythematosus including chilblain lupus erythematosus, rheumatoid arthritis, Sjögren's syndrome, autoimmune nephritis, autoimmune vasculitis, autoimmune hepatitis, autoimmune carditis, autoimmune encephalitis, autoimmune mediated hematological disease, and other autoimmune diseases.

Therapeutic formulations of the active compound, preferably comprising a polypeptide, antibody, oligonucleotide or compound as screened of the invention, are prepared for storage by mixing the active molecule having the desired degree of purity with optional pharmaceutically acceptable carriers, excipients or stabilizers (Remington's Pharmaceutical Sciences 16th edition, Osol, A. Ed. [1980]), e.g. in the form of lyophilized formulations or aqueous solutions.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyl-dimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamin, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In another embodiment, the present invention provides oligonucleotide-based treatment regimens, in particular antisense oligonucleotide-based treatment regimens. He person of skill in the art is aware of how to develop and formulate respective oligonucleotides, such as DNA, RNA (e.g. siRNA), and/or PNA nucleotides. The compositions useful in the treatment of lupus thus include, without limitation, proteins, antibodies, small organic molecules, peptides, phosphopeptides, antisense and ribozyme molecules, triple helix molecules, etc. that inhibit or stimulate the function of TREX1. For example, antisense RNA and RNA molecules act to directly block the translation of mRNA by hybridizing to targeted TREX1-mRNA and preventing protein translation. When antisense DNA is used, oligodeoxyribonucleotides derived from the translation initiation site, e.g., between about −10 and +10 positions of the target gene nucleotide sequence, are preferred. Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4, 469-471 (1994), and PCT publication No. WO 97/33551 (published Sep. 18, 1997).

Nucleic acid molecules in triple helix formation used to inhibit transcription should be single-stranded and composed of deoxynucleotides. The base composition of these oligonucleotides is designed such that it promotes triple helix formation via Hoogsteen base pairing rules, which generally require sizeable stretches of purines or pyrimidines on one strand of a duplex. For further details see, e.g., PCT publication No. WO 97/33551, supra.

These molecules can be identified by any or any combination of the screening assays discussed above and/or by any other screening techniques well known for those skilled in the art.

Lipofections or liposomes can also be used to deliver the TREX1-related molecules into cells. Where antibody fragments are used, the smallest inhibitory fragment which specifically binds to the binding domain of the target protein TREX1 is preferred. For example, based upon the variable region sequences of an antibody, peptide molecules can be designed which retain the ability to bind the target protein sequence. Such peptides can be synthesized chemically and/or produced by recombinant DNA technology (see, e.g., Marascoet al., Proc. Natl. Acad. Sci. USA 90 7889-7893[1993]).

The formulation herein may also contain more than one active compound as necessary for the particular lupus indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active TREX1-related molecules may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes. Sustained-release preparations of the TREX1-related molecules may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y-ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

In yet another embodiment, the present invention concerns a method of treating lupus erythematosus in a human in need thereof, comprising administering to said human a therapeutically effective amount of a compound as identified according to the present invention, or a pharmaceutical preparation according to the present invention. In yet another embodiment, the present invention concerns a method for treating SLE in a human that suffers therefrom comprising administering to the mammal a TREX-1 antisense-nucleic acid molecule, a agonist of a TREX1 polypeptide or an antagonist of a TREX1 polypeptide, wherein said agonist or antagonist preferably is an anti-TREX1 antibody. In another preferred embodiment, the nucleic acid is administered via ex vivo gene therapy. In a further preferred embodiment, the nucleic acid is comprised within a vector, more preferably an adenoviral, adeno-associated viral, lentiviral or retroviral vector.

“Treatment” refers to both therapeutic treatment and prophylactic or preventative measures, wherein the object is to prevent or slow down (lessen) the targeted pathologic condition or disorder, in particular lupus erythematosus. Those in need of treatment include those already with the disorder as well as those prone to have the disorder or those in whom the disorder is to be prevented.

Preferred is a method of treating lupus erythematosus in a human in need thereof according to the present invention, wherein a second immunosuppressive agent is administered. The formulation herein may also contain more than one active compound as necessary for the particular lupus indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended. Administration “in combination with” one or more further therapeutic agents includes simultaneous (concurrent) and consecutive administration in any order.

Most preferred is a method of treating lupus erythematosus in a human in need thereof according to the present invention, wherein said lupus erythematosus is selected from systemic lupus erythematosis, subacute cutaneous lupus erythematosus, cutaneous lupus erythematosus including chilblain lupus erythematosus, rheumatoid arthritis, Sjögren's syndrome, autoimmune nephritis, autoimmune vasculitis, autoimmune hepatitis, autoimmune carditis, autoimmune encephalitis, autoimmune mediated hematological disease, and other autoimmune diseases.

SLE is an autoimmune inflammatory connective tissue disease with variable symptomotolgy including fever, joint pains or arthritis resembling rheumatoid arthritis, erythematous skin lesions and internal organ disease that may lead to cardiac or renal failure. For the past 30 years, cyclophosphamide has been used to treat SLE. Cyclophosphamide however, generally inhibits inflammation and has many side effects. Improvements in the understanding of the molecular events involved in the progression of SLE have lead to the trial of new therapeutic agents with less side effects and more efficacy due to the specific targeting of identified components of SLE. Therapeutics that are immunosuppressive are Mycophenolatemofetil (MMF), Leflunomide, and Tacrolimus (FI506). These molecules have a suppressive effect on the immune system, and in limited trials, have had fewer and less severe side effects than cyclophosphamide.

Biological therapies such as Rituxan', anti-05b, anti-CD40L, anti-BLYS and anti-IL10, are all antibody therapies that have few side effects and target one specific molecule or pathway that is believed to be significant in the progression of SLE. Rituxan, for example, has been used to deplete B cells in SLE patients, and this correlated with improvements in arthritis, rash and fatigue. The biological therapies also have the advantage of being well tolerated, and can be used in combination with established agents such as cyclophosphamide. In the most severe cases of SLE, hematopoeitic stem cell replacement has caused remission in SLE patients, but the long-term results of these cell transplantations has not yet been determined. While cyclophosphamide has remained the major drug treatment of choice for SLE, the new therapies discussed herein that target TREX1 show great promise. The wider range of treatments now in development may mean improved control of this disease.

The compounds of the present invention, e.g., polypeptides, compounds or antibodies, are administered to a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation (intranasal, intrapulmonary) routes. Intravenous or inhaled administration of polypeptides, compounds and antibodies is preferred.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinyl pyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The following examples are offered for illustrative purposes only, and are not intended to limit the scope of the present invention in any way. All patent and literature references cited in the present specification are hereby incorporated by reference in their entirety. The accompanying Figures show:

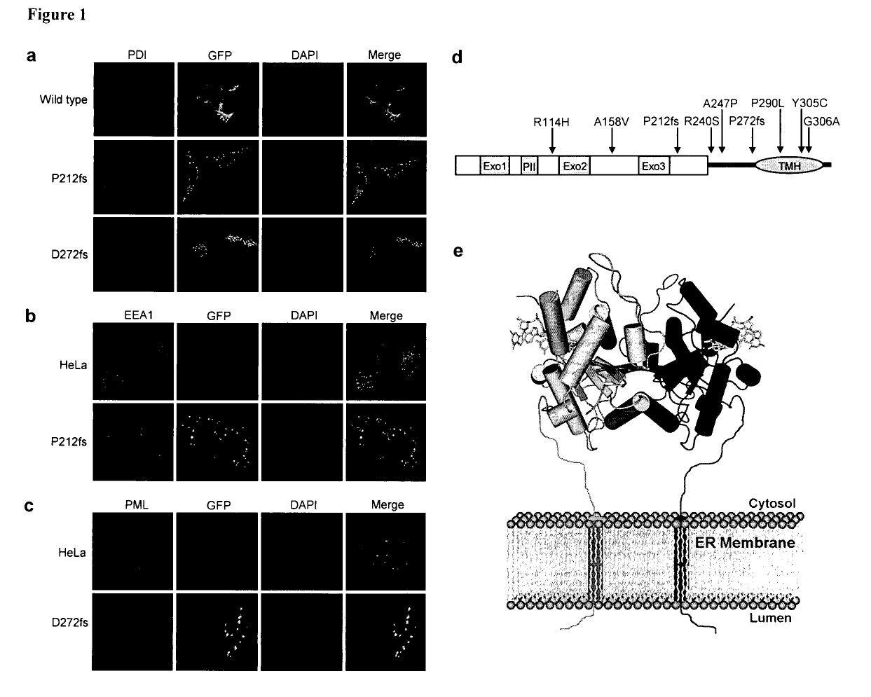

FIG. 1: TREX1 mutations alter subcellular targeting. (a) Subcellular localization of GFP-TREX1-fusion proteins (green, wild type, P212fs, D272fs) in HeLa cells. ER was labelled with anti-PDI antibody (red) and nuclei counterstained with DAPI (blue). (b) HeLa cells stained with anti-EEA1 antibody (red). GFP-TREX1-P212fs co-localizes with endosomal EEA 1 (c) HeLa cells stained with anti-PML antibody (red). Nuclear GFP-TREX1-D272fs co-localizes with PML NBs. (d) Localization of TREX1 mutations. Indicated are the exonuclease domains (Exo1-3), the polyproline II motif (PII) and the transmembrane helix (TMH). (e) Hypothetical model of the TREX1 dimer (monomers in cyan and blue) containing the 2 active sites bound to DNA (yellow). The dimer is connected through a C-terminal linker to the transmembrane region which anchors TREX1 in the ER.

FIG. 2: Multiple protein sequence alignment. Alignment of TREX1 and TREX2 homologs from different species retrieved from the ENSEMBL database (www.ensembl.org) computed with CLUSTAL W (www.ebi.ac.uk/clustalw/). hum: human; chp: chimpanzee; bov: bovine; mse: mouse. The Exo motifs are boxed and indicated. The polyproline II motif is boxed and named PPII, and the C-terminal region unique to TREX1 is also boxed. The four Mg²⁺— coordinating residues are indicated by an arrow. Conserved residues affected by mutations found in patients with SLE or Sjögren's syndrome are marked in gray with the amino acid change indicated above the alignment. The only non-synonymous change that was found also in controls (E226G) is indicated. Residues (S246, S261, S263) of human TREX1 are predicted sites of phosphorylation with a probability of >99% using NetPhos 2.0 (www.cbs.dtu.dk/services/NetPhos/).

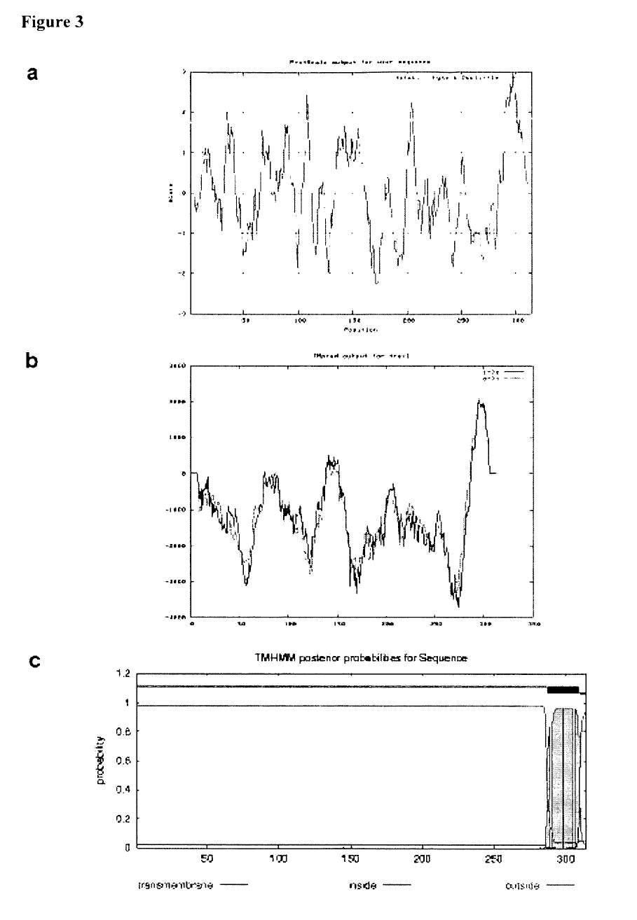

FIG. 3: Supplementary FIG. 2: In silico structural analysis of TREX1 (a) A highly hydrophobic region is revealed by a positive score at the C-terminus corresponding to single transmembrane helix extending over the last C-terminal 30 amino acid residues. The Kyte-Doolittle hydropathy index was computed using the ProtScale tool with the default averaging window size of 9 residues (www.expasy.org/cgi-bin/protscale.pl). Analysis of transmembrane topology with TMpred (www.ch.emnet.org/cgi-bin/TMPRED_from_parser) (b) and TMHMM (www.cbs.dtu.dk/cgi-bi) (c) was in agreement with ProtScale and also predicted a transmembrane helix.

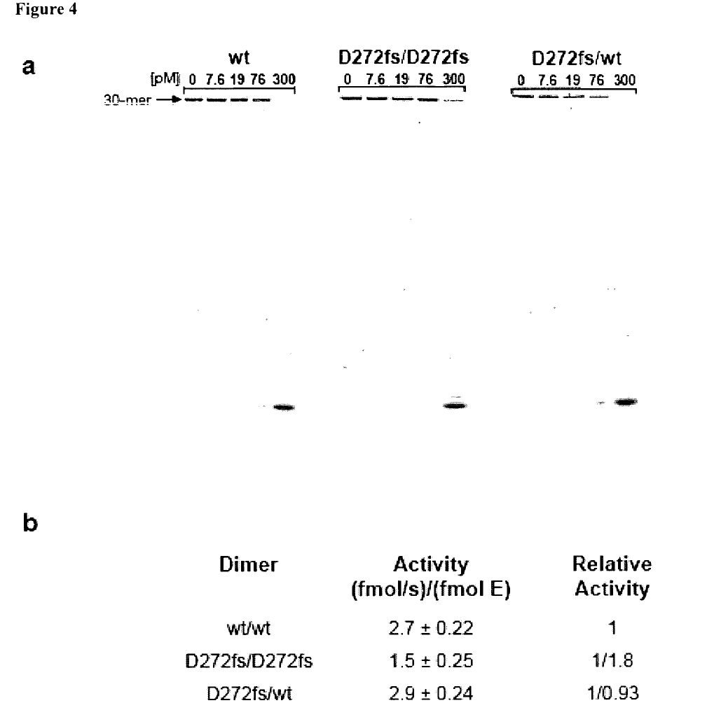

FIG. 4: Supplementary FIG. 3: Exonuclease activity of recombinant TREX1 (a) Enzyme activity of increasing amounts of recombinant wild type (wt) and mutant (D272fs) TREX1 homo- and heterodimers. (b) To more precisely quantify the relative exonuclease activities of the D272fs/D272fs and D272fs/wt dimers compared to wt/wt dimers each enzyme was assayed in triplicate at three different concentrations (38, 57, and 76 μM) as described above. Plots of activity vs. enzyme concentrations were used to confirm linearity of the assay and to generate the enzyme activity values. The average activities and standard errors for TREX1, D272fs/D272fs, and D272fs/wt were determined by regression analysis using SigmaPlot 8.02 (SPSS Science, Inc.). The relative activity was calculated as: Relative activity=100×[(fmol of dNMP released/s/fmol of mutant enzyme)/(fmol of dNMP released/s/fmol wt enzyme)].

SEQ ID No. 1 shows the genomic nucleic acid sequence of trex1.

SEQ ID No. 2 shows the amino acid sequence of TREX1.

SEQ ID No. 3 shows the nucleic acid sequence of trex1-cDNA.

SEQ ID Nos. 4 to 9 show primers for amplifying the coding exon and part of the 3′UTR of the TREX1 gene by 3 overlapping PCR amplicons.

### EXAMPLES

### Methods

### Samples

All participating individuals gave written informed consent and DNA was collected in compliance with the principles of the Helsinki Accord. The SLE cohorts from the UK and Finland were collected as previously described (Aitman, T. J. et al. Copy number polymorphism in Fcgr3 predisposes to glomerulonephritis in rats and humans. Nature 439, 851-855 (2006), Koskenmies, S., Widen, E., Kere, J., and Julkunen, H. Familial systemic lupus erythematosus in Finland. J. Rheumatol. 28, 758-760 (2001)). The German SLE and Sjögren's syndrome cohorts were established as part of the study “Genetic Risk Factors of SLE” and the research was approved by the Medizinische Hochschule Hannover Ethics Committee. Except for the Finnish cohort which consisted of nuclear family trios (both parents and one affected child with SLE), all other cohorts consisted of unrelated cases and controls. SLE and Sjögren's syndrome phenotypes were defined according to the diagnostic criteria by the American Rheumatism Association and the international consensus criteria for Sjögren's syndrome (Tan, E. M. et al. The 1982 revised criteria for the classification of systemic lupus erythematosus. Arthritis Rheum. 25, 1271-1277 (1982); Vitali, C. et al. Classification criteria for Sjögren's syndrome: a revised version of the European criteria proposed by the American-European Consensus Group. Ann. Rheum. Dis. 61, 554-558 (2002)), respectively. In total n=417 cases with SLE and 169 cases with Sjögren's syndrome were analysed. 85.8% of SLE cases from the UK were of Caucasian ethnicity. 7.1% were of Indian, 4.5% of Afro-Caribbean, and 2.6% of either Oriental, mixed, or Filipino decent. The 200 UK controls were taken from the “Genetic mechanisms of cardiovascular disease cohort” at the University of Glasgow. German control samples were obtained from the blood donation program at the Medizinische Hochschule Hannover and the Universitätsklinikum Dresden. In total n=1512 German control samples were analysed. In addition, 188 Finish nuclear family trios were analysed.

### Mutation Analysis

The entire coding exon and part of the 3′UTR of the TREX1 gene were amplified by 3 overlapping PCR amplicons from genomic DNA extracted from peripheral lymphocytes. All 3 amplicons were generated in >99% of samples.

The following primers were used:

(SEQ ID No. 4)
TREX1-1s: 3′-CCTCCCCTTCGGATCTTAAC-5′
and
(SEQ ID No. 5)
TREX1-1as: 3′-CCCATACAGGCGAGTGTAGA-5′,
(SEQ ID No. 6)
TREX1-1cs: 3′-GGGCGTCAATGTTTTGATGA-5′
and
(SEQ ID No. 7)
TREX1-1cas: 3′-GAGGCTGTGACCCCATACAT-5′,
(SEQ ID No. 8)
TREX1-1ds: 3′-ACTCGCCTGTATGGGCAGTC-5′
and
(SEQ ID No. 9)
TREX1-1das: 3′-GGGAAAGTGAGGGACAAACA-5′.

Purified PCR products were sequenced using fluorescent dye terminator chemistry on ABI 3730 instruments. Both strands of the amplicons were sequenced to obtain unambiguous sequence reads. >99% of sequencing reads passed quality filters and had unambiguous sequence results. The percentage of PCR and sequencing failures was the same for cases and controls. The total number of samples that was analysed in the final dataset is as presented above. All identified variants were re-sequenced and validated independently by an additional sequencing method (pyrosequencing). All variants with non-synonymous changes were sub-cloned from genomic DNA of individual patients. Sequence reads were processed and assembled using Phred and Phrap, and analysed using Polyphred and Consed (http://www.phrap.org/; http://droog.mbt.washington.edu/PolyPhred.html). All newly identified SNPs have been submitted to dbSNP.

### Statistical Analysis

The numbers of mutations or variants between cases and controls were compared with a two by two contingency table using a two-sided Fischer's exact test (www.r-project.org).

### Preparation of Homo- and Heterodimeric Mutant Trex1 Enzymes

For preparation of homodimers the human TREX1 gene was expressed as a fusion with the maltose binding protein (MBP) in pLM303, a pET-27b (Novagen) derivative encoding a polyhistidine sequence on the N-terminus of MBP and a rhinovirus 3C protease recognition site between the MBP and TREX1 genes. The plasmid was transformed into E. coli BL21(DE3) Rosetta 2 cells (Novagen) for overexpression. The cells were grown to an O.D. 600 ˜0.5 at 37° C. and quickly cooled on ice to 17° C. After induction with 1 mM isopropyl-β-D-thiogalacto-pyranoside (IPTG) the cells were allowed to grow for 15 h at 17° C. The MBP-TREX1 fusion protein was purified by affinity chromatography using an amylose resin (New England Biolabs). The MBP protein was removed from the fusion by treatment with PreScission Protease™ (GE Biosciences) at 4° C. for 20 h. The TREX1 homodimer protein was separated from the MBP by chromatography using a phosphocellulose column.

To prepare TREX1 heterodimers, a mutant (mut) and a wild-type (wt) copy of TREX1 were cloned on separate plasmids for co-expression in E. coli. First, the pLM303 plasmid was modified to eliminate the polyhistidine sequence on the N-terminus of MBP, and the TREX1 mutant genes were cloned into the resultant modified vector pLM303X retaining the rhinovirus 3C protease recognition site between MBP and TREX1. A second plasmid containing the wt TREX1 gene was prepared as a fusion of the His-tagged NusA with TREX1 in the pCDFDuet-1 plasmid (Novagen). The pLM303X-TREX1mut and pCDFDuet-1-TREX1-wt plasmids were co-expressed in E. coli BL21(DE3) Rosetta 2 cells (Novagen) resulting in the formation of 1) wt/wt homodimers, 2) wt/mut heterodimers, and 3) mut/mut homodimers. The His-tagged NusATREX1 wt/His-tagged NusATREX1 wt homodimers and His-tagged NusATREX1 wt/MBPTREX1mut heterodimers were recovered by affinity chromatography using a nickel-NTA resin (Qiagen). The His-tagged NusATREX1 wt/MBPTREX1 mut heterodimer was purified by affinity chromatography using an amylose resin. The His-tagged NusA and MBP proteins were removed from the fusion by treatment with PreScission Protease™ (GE Biosciences) and purified using a phosphocellulose column.

### Exonuclease Assay

Enzyme activities were determined as described previously (Mazur, D. J. and Perrino, F. W. Identification and expression of the TREX1 and TREX2 cDNA sequences encoding mammalian 3′-->5′ exonucleases. J. Biol. Chem. 274, 19655-19660 (1999). Mazur, D. J. and Perrino, F. W. Excision of 3′ termini by the Trex 1 and TREX2 3′-->5′ exonucleases. Characterization of the recombinant proteins. J. Biol. Chem. 276, 17022-17029 (2001).). Briefly, exonuclease reactions (30 μl) were prepared with a PAM-labeled ssDNA 30-mer oligonucleotide substrate and dilutions of the TREX1 enzymes were prepared at 10 times the final concentrations. Samples (3 μl) of the indicated TREX1 enzymes were added to reactions to generate the indicated final enzyme concentration. Incubations were 20 min at 25° C. Reaction products were subjected to electrophoresis on 23% polyacrylamide gels.

### Transfection and Immunofluorescence

5×10⁴ HeLa cells were grown on cover slips and transfected with GFP-TREX1-fusion constructs cloned into pEGFP-C1 using Fugene 6 (Roche). Cells were fixed with 3.7% paraformaldehyde, permeabilized with 0.1% Triton-X, and incubated with mouse anti-protein disulfide isomerase (Stressgen), mouse anti-PML (Santa Cruz), or rabbit anti-EEA1 (Calbiochem) antibody, respectively, followed by incubation with Alexa Fluor 594-labeled goat anti-mouse or goat anti-rabbit IgG, respectively. Cover slips were mounted in Vectashield containing DAPI (Vectorlabs). Confocal microscopy was done with a Zeiss LSM510 laser scanning microscope and images processed using the LSM Image Browser.

TREX1 was identified in a German family showing clinical phenotypes that were consistent with the diagnosis of chilblain lupus, a rare cutaneous form of lupus erythematosus (Hutchinson J (1888) Harveian lectures on lupus. On the various forms of lupus vulgaris and erythematosus. British Medical Journal 1, 118). The presence of histological and serological findings typically seen in lupus constitute diagnostic criteria and differentiates chilblain lupus from true cold induced perniosis or chilblains (Franceschini, F., Calzavara-Pinton, P., Quinzanini, M., Cavazzana, I., Bettoni, L., Zane, C., Facchetti, F., Airo, P., McCauliffe, D. P., Cattaneo, R. (1999) Chilblain lupus erythematosus is associated with antibodies to SSAIRo. Lupus 8, 215-219). It occurs predominantly in adult women and has only rarely been described in children (Millard, L. G., Rowell, N. R. (1978) Chilblain lupus erythematosus (Hutchinson). A clinical and laboratory study of 17 patients. Br. J. Dermatol. 98, 497-506; Weston, W. L., Morelli, J. G. (2000) Childhood pemio and cryoproteins. Pediatr. Dermatol. 17, 97-99). Apart from two affected brothers, no familial occurrence has been described to date (Weston, W. L., Morelli, J. G. (2000) Childhood pemio and cryoproteins. Pediatr. Dermatol. 17, 97-99). Progression to systemic lupus erythematosus has been reported in up to 18% and is thus, comparable to the progression rate of classic chronic cutaneous lupus erythematosus (Millard, L. G., Rowell, N. R. (1978) Chilblain lupus erythematosus (Hutchinson). A clinical and laboratory study of 17 patients. Br. J. Dermatol 98, 497-506).

The vertical transmission of the disease in both males and females and the consistency of the phenotype suggested autosomal dominant inheritance with high penetrance. The phenotype of all family members was established through direct clinical exam or review of medical records. All participating individuals granted informed written consent prior to the study. In order to map the disease locus, whole-genome linkage analysis was carried out on genomic DNA from peripheral blood leukocytes from 26 family members including 14 affected individuals using the Affymetrix GeneChip Human Mapping 10K Xba 142 2.0 Array. Genotyping of microsatellite markers was carried out with fluorescently labeled primers on an ABI3100 Sequencer (Applied Biosystems). GRR and PedCheck were used to verify relationship and gender and to identify Mendelian errors (Abecasis, G. R., Chemy, S. S., Cookson, W. O., Cardon, L. R. (2001) GRR: graphical representation of relationship errors. Bioinformatics. 17, 742-743; O'Connell, J. R., Weeks, D. E. (1998) PedCheck: a program for identification of genotype incompatibilities in linkage analysis. Am. J. Hum. Genet. 63, 259-266). Non-parametric and parametric linkage analysis was carried out with Merlin 1.0 (Abecasis, G. R., Chemy, S. S., Cookson, W. O., Cardon, L. R. (2002) Merlin—rapid analysis of dense genetic maps using sparse gene flow trees. Nat. Genet. 30, 97-101). For parametric analysis a fully penetrant autosomal dominant trait without phenocopy and a disease allele frequency of 0.001 were assumed.

The disease locus was localized on chromosome 3p21-3p14 with a maximum LOD score of 5.04 (FIG. 4). Non-parametric analysis confirmed this locus with a Z-mean score of 6.2. A common haplotype, that was shared by all affected and absent in all unaffected individuals, was identified. Based on critical recombination events in 2 affected individuals the disease interval was defined to a 13.8 cM region delimited by markers rs704920 and d3s1300 (FIG. 4). The mapped interval comprises 16.4 Mb and includes over 100 known genes (Ensembl v38).

By systematic mining of the Ensembl and GeneCards databases the function and expression of all genes were investigated and candidate genes selected for sequencing. All coding exons including flanking intronic regions were sequenced on an ABI3100 sequencer according to standard procedures. Priority was given to genes that had been implicated in the pathogenesis of lupus erythematosus or other autoimmune diseases such as the genes encoding toll like receptor 9, [TLR9, MIM *605474] deoxyribonuclease I-like 3 [DNASE1L3, MIM *602244], ubiquitin-activating enzyme E1-like [UBE1L, MIM *191325], protein kinase C delta [PRKCD, MIM *176977], interleukin 17 receptor B and D [IL17RB, MIM *605458; IL17RD, MIM *606807], and the chemokine receptor cluster [CCR9, MIM *604738; CXCR6, MIM *605163; XCR1, MIM *600552; CCR1, MIM *601159; CCR2, MIM *601267; CCR3, MIM *601268; CCR5, MIM *601373; CCRL2, MIM *608379). The inventors also investigated the collagen VII alpha-1 gene [COL7A1, MIM *120120], since it is highly expressed in the skin and known to cause autosomal dominant bullous epidermolysis dystrophica [MIM *131750], although blistering is not a feature of chilblain lupus. Several known and novel SNPs were identified, but no pathogenic mutation was observed within the regions analyzed. All novel coding SNPs were interrogated for segregation with the trait and their allele frequencies estimated by genotyping 100 unrelated healthy controls (data not shown).

Comparative sequencing of the Trex 1 gene located within the mapped disease-haplotype revealed a heterozygous change at position 20570 of the genomic sequence (g.20570G>A), that leads to the exchange of an aspartic acid with an asparagine residue (D18N). This mutation was present in all family members affected with chilblain lupus and absent in all non-affected members. The mutation lies within the first of three EXO domains, EXO I, of the Trex1 gene, which generate the exonuclease active site of the Trex1 enzyme. The highly conserved EXO motifs are characteristic features of a number of exonucleases from different species including the proofreading exonucleases of bacterial replicative DNA polymerases (Mazur et al. Identification and expression of the Trex1 and Trex2 cDNA sequences encoding mammalian 3″-5′ exonucleases, J. Biol. Chem. 1999, 274, 19655-19660, Höss et al. A human DNA editing enzyme homologue to the Escherichia coli DnaQ/MutD protein, EMBO 1999, 18, 3868-3875). Within these EXO domains, 4 amino acid residues, an aspartic acid and a glutamic acid contributed by EXO 1 and 2 aspartic acid residues contributed each by the EXO II and EXO III domain, respectively, are critical for metal ion binding and show the highest degree of conservation. The mutation D18N affects the first highly conserved aspartic acid within the EXO I motif and is, thus, predicted to impede enzymatic activity. Analysis of enzymatic activity of the Trex1 mutant was performed in nuclear extracts of Epstein Barr-virus-transformed lympholastoid cells from 3 affected individuals as described by Morita et al. (Morita et al. (2004) Gene-targeted mice lacking the Trex1 (DNase III) 3′-5′DNA exonuclease develop inflammatory myocarditis. Mol. Cell. Biol. 24, 6719-6727). Enzyme assays were performed by incubation of purified nuclear extracts with poly(dA) substrate, which was 3′-end labeled by the incorporation of [³²P]dAMP by terminal transferase. The substrate was ethanol precipitated in the presence of denatured calf thymus carrier DNA and the radioactive material released into the supernatant determined by scintillation counting. Enzymatic activity was found to be reduced to up to 50% in affected individuals as compared to unrelated healthy control samples suggesting that the mechanism underlying the phenotype may be mediated by haploinsufficiency of the Trex1 gene.

Because of the phenotypic overlap of chilblain lupus with common forms of lupus erythematosus the inventors tested the hypothesis that patients with sporadic forms of systemic lupus erythematosus carry mutations within the Trex1 gene. Using a case-control approach a cohort of 90 unrelated patients with systemic lupus erythematosus diagnosed according to the criteria of the American Rheumatic Association was compared with 96 unrelated healthy individuals by direct sequencing of all exons including flanking intronic regions of the Trex1 gene. Among patients with Lupus erythematodus, first, six mutations, five missense and one frame shift mutation, were identified, none of which was found in healthy controls (as shown in Table 1). In addition, several genetic variants were found, both within non-coding exonic and intronic regions. Of those 11 were present only among patients with SLE and not in healthy controls, and four were found to occur at different frequencies (Table 1). This first set of data shows that genetic variants of the Trex1 gene occur more frequently among patient with lupus erythematosus suggesting that changes in the protein and/or the regulation of its transcription may play a causal role in the pathogenesis of lupus erythematosus.

The inventors further sequenced the coding region of TREX1 in four cohorts from UK, Germany, and Finland. The inventors identified five heterozygous missense and one frameshift change in 6/218 SLE cases from the UK compared with 0/200 non-synonymous changes in controls (P=3.1×10⁻²) (see Table 2). All missense changes affect highly conserved residues except one (E266G) (FIG. 2). In the German SLE cohort, four heterozygous missense changes, one frameshift and a single 3′UTR variant were found in 6/199 cases compared to 2/1512 controls (P=5.3×10⁻⁵) (Table 2). Among these four non-synonymous changes, two alter highly conserved residues, while two patients carried the non-conserved variant E266G. Similarly, the only non-synonymous change identified in two German controls was E266G. Moreover, the inventors screened 169 German patients with primary Sjögren's syndrome and identified one additional missense change in a completely conserved residue that was not observed in any of the control populations studied (FIG. 2). The inventors furthermore sequenced TREX1 in 188 Finish nuclear family trios and did not detect any non-synonymous change in these 564 individuals. The absence of non-synonymous changes in this cohort may be explained by the different demographic history of the Finnish population (Laan, M. and Paabo, S, Nat. Genet. 17, 435-438 (1997)).

TABLE 2
Frequency of TREX1 mutations

| | Number of mutations | Number of mutations | |
|---|---|---|---|
| Cohort | in patients | in controls | P-value |
| SLE | 12/417 | 2/1712 | 1.7 × 10⁻⁷ |
| a) UK | 6/218 | 0/200 | 3.1 × 10⁻² |
| b) Germany | 6/199 | 2/1512 | 5.3 × 10⁻⁵ |

If only sequence variants are grouped that are not present in controls the significance level in the combined dataset would change to (10/417 vs. 0/1712) P = 8.0 × 10⁻⁸.

In addition, 13 synonymous variants were identified in TREX1 in the entire dataset (Table 3). All variants, except one, occurred at a very low allele frequency between 0.01 and 1.9%, and were found at similar frequencies among cases and controls across the different populations tested.

TABLE 3
Polymorphisms in the TREX1 gene II (cf. also table 1)

| | | | Major/ | Minor |
|---|---|---|---|---|
| | Accession | Position on | minor | allele |
| SNP | number | chromosome 3 | allele | frequency |
| TREX1- | ss71651743 | 48448241 | G/A | 0.005 |
| 20701 G > A | | | | |
| TREX1- | ss71651744 | 48448247 | G/A | 0.001 |
| 20707 G > A | | | | |
| TREX1- | rs3135943 | 48448256 | G/A | 0.003 |
| 20716 G > A | | | | |
| TREX1- | ss71651745 | 48448370 | G/C | 0.002 |
| 20830 G > C | | | | |
| TREX1- | ss71651746 | 48448397 | G/A | 0.005 |
| 20857 G > A | | | | |
| TREX1- | ss71651747 | 48448454 | C/T | 0.001 |
| 20914 C > T | | | | |
| TREX1- | rs3135944 | 48448520 | T/C | 0.003 |
| 20980 T > C | | | | |
| TREX1- | ss71651748 | 48448544 | G/A | 0.002 |
| 21004 G > A | | | | |
| TREX1- | rs11797 | 48448589 | C/T | 0.425 |
| 21049 C > T | (see also | | | |
| | table 1) | | | |
| TREX1- | ss71651749 | 48448748 | G/A | 0.003 |
| 21208 G > A | | | | |
| TREX1- | ss71651750 | 48448855 | A/G | 0.001 |
| 21315 A > G | (see also | | | |
| (E266G) | table 1) | | | |
| TREX1- | rs3135945 | 48448970 | G/A | 0.019 |
| 21430 G > A | (see also | | | |
| | table 1) | | | |
| TREX1- | rs3135946 | 48449040 | T/C | 0.006 |
| 21500 T > C | (see also | | | |
| | table 1) | | | |
| TREX1- | ss71651751 | 48449061 | C/T | 0.001 |
| 21521 C > T | | | | |

Positions of SNPs are indicated according to NCBI Build Hs36.2_chr3, NT_22517.17. Minor allele frequency as determined in 2298 individuals (586 patient and 1712 control samples). All alleles were observed at similar frequencies in cases and controls. All variants are synonymous substitutions except TREX1-21315 A > G which results in the amino acid substitution E266G.

Taken together, with respect to the preferred mutations as shown in table 2, the inventors observed one 3′UTR variant and 11 non-synonymous changes in 417 patients with SLE (2.64%), but only two non-synonymous changes in 1712 controls (P=1.7×10⁻⁷). The inventors' combined data identify a strongly increased relative risk for development of SLE in patients that carry TREX1 variants (RR=25.3; 95% CI 5.6-232.4). The phenotypic features of SLE patients carrying TREX1 mutations encompass a broad clinical spectrum of the disease including symptoms of Sjögren's syndrome (Table 4). This observation prompted the inventors to investigate TREX1 in a patient cohort with primary Sjögren's syndrome and the inventors found one non-synonymous mutation that was not detected in 1712 controls, raising the possibility of a commonly underlying genetic risk for these systemic autoimmune diseases.

Five missense changes found in patients are located within the C-terminus outside the catalytical domains, while the two frameshift mutations, P212fs and D272fs, result in C-terminally truncated proteins (FIG. 1 and FIG. 2). Among the unique features of TREX1, not present in the homologous TREX2, is a highly conserved C-terminal extension of 79 amino acids with no known homology (Mazur, D. J. and Perrino, F. W. J. Biol. Chem. 274, 19655-19660 (1999)) (FIG. 1). In silico analysis predicted a single transmembrane helix involving residues 286-309 (FIG. 3). Since a truncated version of TREX1 (residue 1-242) is nearly fully capable of catalysis, the inventors predicted that these mutations would not greatly affect enzyme activity. Exonuclease activity of recombinant D272fs/D272fs homodimers and D272fs/wt heterodimers were 56% and 107%, respectively of wt homodimers (FIG. 4). The inventors' in-vitro findings suggest that no major enzyme deficiency is caused by D272fs.

The inventors next examined whether the frameshift mutations that eliminate the putative transmembrane domain might alter TREX1 subcellular localization using N-terminal GFP-TREX1-fusion constructs. In HeLa cells, wild type GFP-TREX1 associated with the ER, as shown by co-localization with protein disulfide isomerase (FIG. 1). GFP-TREX1-P212fs, in which the last 103 amino acids are replaced by a 64 residue aberrant peptide, distributed in a punctate pattern throughout the cytoplasm. Co-staining with early endosomal antigen 1 confirmed uptake into endosomal vesicles (FIG. 1). In contrast, GFP-TREX1-D272fs, which lacks the last 42 residues, was almost exclusively localized within the nucleus (FIG. 1), and distributed in a speckled pattern within the interchromatin space suggesting association with subnuclear organelles (FIG. 1). PML (promyelocytic leukemia protein) is a major component of PML nuclear bodies (PML NBs), dynamic structures that act as sensors of DNA damage and recruit ssDNA following exposure of cells to UV-light (Boe, S. O. et al. J. Cell Sci. 119, 3284-3295 (2006)). Moreover, PML is induced by interferons and its expression required for interferon-induced apoptosis and antiviral host defense (Regad, T. and Chelbi-Alix, M. K. Oncogene 20, 7274-7286 (2001)). Co-staining for PML revealed that GFP-TREX1-D272fs co-localizes with PMI, NBs (FIG. 1). Thus, both frameshift mutations alter the subcellular distribution of TREX1 and might thereby interfere with its function. The crystal structure of TREX1 (residue 1-242) identified a polyproline II helix (FIG. 1) (de Silva, U. et al. J. Biol. Chem. 282, 10537-10543 (2007)) known sites of protein-protein interactions. It is possible that the identified missense mutations might interfere with exonuclease activity, subcellular targeting or interactions with other proteins. Moreover, variation in the 3′UTR could result in altered transcript levels (Mendell, J. T. and Dietz, H. C. Cell 107, 411-414 (2001)).

The inventors' in-vitro assays suggest that the C-terminus is required for cellular compartmentalization of TREX1. Based on these findings, the inventors propose that each TREX1 monomer is anchored in the ER membrane via the C-terminal transmembrane domain (FIG. 1). The connecting linker region which contains multiple predicted sites of likely Ser phosphorylation (FIG. 2) may be subject to post-translational modification in response to oxidative stress, allowing nuclear translocation of TREX1. In this model, the active sites of each monomer are readily accessible to ssDNA (de Silva, U. et al. J. Biol. Chem. 282, 10537-10543 (2007)).

Viral infection has been implicated as an environmental factor in the pathogenesis of SLE and Sjögren's syndrome (James, J. A., Harley, J. B., and Scofield, R. H. Curr. Opin. Rheumatol. 13, 370-376 (2001)). Viral infection triggers immune responses via recognition of viral DNA through TLR9 in endosomes. Yet, cytosolic DNA can induce a TLR9-independent type I interferon immune response, suggesting the existence of a distinct cytosolic DNA sensor (Stetson, D. B. and Medzhitov, R. Immunity 24, 93-103 (2006)). By virtue of its high affinity for ssDNA and cytosolic localization, TREX1 may be a plausible candidate for this role. Further studies looking at induction of interferon responsive genes in cells in which TREX1 is silenced are necessary to investigate this hypothesis.

TREX1 is not the first DNase found to be associated with SLE. Mutations in DNase I, the most abundant extracellular DNase, cause SLE in humans and mice (Napirei, M. et al. Nat. Genet. 25, 177-181 (2000), Yasutomo, K. et al. Nat. Genet. 28, 313-314 (2001)) and DNase II^{-/-} mice develop a rheumatoid arthritis-type syndrome (Kawane, K. et al. Nature 443, 998-1002 (2006)). A dual role of TREX1 as DNA degrading enzyme in granzyme A-mediated apoptosis and potentially as cytosolic DNA sensor may explain how impaired TREX1 function could induce an environment in which autoimmunity is perpetually stimulated and sustained.

TABLE 4
TREX1 mutations and phenotypic features of patients with SLE or Sjögren's syndrome

| Patient | nucleotide | amino acid | ethnic origin | sex | phenotype |
|---|---|---|---|---|---|
| 1 | 341G > A | R114H | European | F | nephritis, arthritis, ANA, anti-dsDNA |
| 2 | 473C > T | A158V | European | F | photosensitivity, Raynaud's, ANA, anti-dsDNA |
| 3 | 634delC | P212fs | European | F | discoid rash, photosensitivity, antiphospholipid syndrome, anti-Ro/SSA |
| 4 | 679G > A + | G227S + | Afro-Caribbean | F | nephritis, pleurisy, oral ulcers, arthritis, Sjögren's |
| | 739G > C | A247P | | | |
| 5 | 720G > C | R240S | Afro-Caribbean | F | malar rash, oral ulcers, Raynaud's, Sjögren's, ANA, anti-Ro/SSA |
| 6 | 812\hat{ }813insAA | D272fs | European | F | nephritis, oral ulcers, photosensitivity, Raynaud's |
| 7 | 869C > T | P290L | European | M | discoid rash, arthritis, photosensitivity, ANA, anti-ssDNA |
| 8 | 914A > G | Y305C | European | F | discoid rash, arthritis, antiphospholipid syndrome, thrombocytopenia, ANA |
| 9 | 917G > C | G306A | European | F | Sjögren's, anti-Ro/SSA |
| 10 | 979delC | 3′UTR | European | F | discoid rash, malar rash, arthritis, autoimmune hepatitis, ANA |

cDNA and amino acid numbering is based on NCBI accession numbers AF319569 and AAK07616, respectively, with the A of the initiating ATG codon as the first base. F: female; M: male; ANA: antinuclear antibodies; Raynaud's: Raynaud's disease; Sjögren's: Sjögren's syndrome. None of the reported nucleotide changes occurred in 1712 control samples (i.e. 3424 alleles). G227S and A247P were found on the same allele. All mutations are novel, except for R114H, which is commonly found in patients with AGS1.

1-23. (canceled)

24. A method of diagnosing lupus erythematosus in a human, said method comprising detecting a genetic variant in the gene encoding for TREX1 in a sample from a subject, wherein said genetic variant is a mutation or polymorphism, and wherein said genetic variant indicates the subject has or is at risk of having lupus erythematosus.

25. The method of claim 24, wherein said diagnosis further includes the step of an analysis of the risk to develop a lupus erythematosus or an analysis of the risk for a relapse of lupus erythematosus in said human.

26. The method of claim 24, wherein said method comprises the steps of:
a) using an automated sequencing instrument to sequence nucleic acid encoding TREX1 from a biological test sample obtained from a human to be diagnosed;
b) comparing the sequence obtained in (a) with a sequence of the gene encoding TREX1 in a control sample from a healthy individual, wherein a difference between the sequence of the gene encoding TREX1 in said human to be diagnosed and the TREX1 sequence for said control sample identifies a genetic variant and diagnoses lupus erythematosus.

27. The method of claim 26, wherein said genetic variant detected by sequencing of nucleic acid encoding TREX1 is found in a regulatory region, intron, exon, or coding exon 11 of TREX1, or in a sequence as set forth in SEQ ID NO: 1.

28. The method of claim 26, wherein said genetic variant detected by sequencing of nucleic acid encoding TREX1 is found in sequence encoding the C-terminal 150 amino acids of the human TREX1 protein, or in at least one conserved region of the gene encoding for TREX1.

29. The method of claim 26, wherein the genetic variant detected by sequencing of nucleic acid encoding TREX1 is selected from a variant at position 114, 158, 212, 227, 240, 247, 272, 290, 305 and 306 of the human TREX1 protein as set forth in SEQ ID NO: 2, or directly adjacent amino acid positions, or the homologous positions thereof in a TREX1 protein from another species.

30. The method of claim 26, wherein the genetic variant detected by sequencing of nucleic acid encoding TREX1 is selected from the group of polymorphisms consisting of g.3829A>T; g.3858A>G; g.4032A>C, rsl 1925638; g.H 170OT, rs35240314; g.14283G>A; g.14384OT; g.14455T>C; g.17696OT, rs34426134; g. 18682OA, rs3135936; g.18743G>A; g.18853A>T; g.20131T>C, rs3135941; g.20570G>A; g.20852OA; ss71651743, 20701 G>A; ss71651744, 20707 G>A; rs3135943, 20716 G>A; ss71651745, 20830 G>C; ss71651746, 20857 G>A; ss71651747, 20914 OT; rs3135944, 20980 T>C; ss71651748, 21004 G>A; g.21049C>T, rsl 1797; g.21 197G>A; ss71651749, 21208 G>A; g.21238G>C; g.21257G>C; g.21315A>G; g.21330Λ 21331insAA; g.21430G>A, rs3135945; g.21500T>C, rs3135946; and ss71651751, 21521 C>T.

31. The method of claim 24, wherein detection of a genetic variant comprises at least one of nucleic acid hybridization, restriction enzyme digestion analysis, and nucleic acid sequencing analysis.

32. The method of claim 24, further comprising the analysis of one or more additional genetic markers for lupus erythematosus, selected from the group consisting of MICA, CTLA4, CD 154 (3′UTR), PDCD1, RNUX1, PTP22, SCL22A4, SUMO4, and IRF5.

33. The method of claim 24, wherein lupus erythematosus is selected from systemic lupus erythematosis, subacute cutaneous lupus erythematosus, cutaneous lupus erythematosus including chilblain lupus erythematosus, rheumatoid arthritis, Sjögren's syndrome, autoimmune nephritis, autoimmune vasculitis, autoimmune hepatitis, autoimmune carditis, autoimmune encephalitis, and autoimmune mediated hematological disease.

34. The method of claim 24, wherein said method further includes the step of selecting measures for secondary or primary prevention based on said diagnosis.

35. A method of diagnosing lupus erythematosus in a human, said method comprising measuring the expression of the gene encoding TREX1 in a sample and comparing the measured expression of a standard, wherein a difference is indicative of the risk or presence of lupus erythematosus.

36. The method of claim 35, wherein detection of said change of expression comprises RT-PCR amplification or contacting a sample with an antibody directed to TREX1.

37. The method of claim 36, wherein said detection of change of expression of the gene encoding TREX1 comprises contacting a sample with an antibody directed to TREX1, permitting said antibody to bind TREX1 polypeptide in said sample, and determining the binding of said antibody to said sample.

38. The method of claim 37, wherein said determination of binding is analyzed using a light microscope, flow cytometer, or a fluorimeter.

39. The method of claim 37, wherein said antibody carries a detectable label.

40. The method of claim 37, wherein said antibody is polyclonal, monoclonal, humanized, bispecific, or heteroconjugate.

41. A method of diagnosing lupus erythematosus in a human, said method comprising detecting a change in the biochemical activity of TREX1 wherein a difference in biochemical activity is indicative of the risk or presence of lupus erythematosus.

42. The method of claim 41, wherein detection of said change of biochemical activity comprises a 3′ nucleotide excision assay, wherein said 3′ nucleotide excision assay comprises contacting a DNA substrate with a labeled extract comprising a TREX1 protein isolated from a human being at risk or having lupus erythematosus, under conditions that permit TREX1 to excise a nucleotide from said substrate.

43. A method of identifying a compound that modifies the activity of TREX1, said method comprising contacting cells expressing TREX1 with a candidate compound, and determining the activity of TREX1 in response to said candidate compound.

44. The method of claim 43, wherein said activity of TREX1 being modified by said compound is selected from the biochemical activity or the expression of the gene encoding for TREX1.

45. The method of claim 45, wherein said determining of said modification of TREX1 activity comprises using an antibody directed against TREX1, RT-PCR amplification, or a 3′ nucleotide excision assay.

46. The method of identifying a compound that modifies the activity of TREX1 according to claim 45, wherein said candidate compound is a TREX1 antisense nucleic acid.

47. A diagnostic kit, comprising: a container comprising materials for performing a diagnostic method according to claim 24, together with suitable agents and instructions for use.

48. A method of treating lupus erythematosus in a human, the method comprising the steps of:
a) using an automated sequencing instrument to sequence nucleic acid encoding TREX1 from a biological test sample obtained from a human to be diagnosed;
b) comparing the sequence obtained in (a) with a sequence of the gene encoding TREX1 in a control sample from a healthy individual, wherein a difference between the sequence of the gene encoding TREX1 in said human to be diagnosed and the TREX1 sequence for said control sample, identifies a genetic variant and indicates that said human to be diagnosed has lupus erythematosus; and
c) if lupus erythematosus is diagnosed, administering an effective amount of a therapeutic agent for treating lupus erythematosus.

49. The method of claim 48, further comprising administering an immunosuppressive agent in addition to said therapeutic agent.
